(19) United States
(12) Patent Application Publication
(10) Pub. No.: US 2005/0019836 A1
(43) Pub. Date: Jan. 27, 2005

(54) Bioanalytical reagent, method for production thereof, sensor platforms and detection methods based on use of said bioanalytical reagent

(76) Inventors: Horst Vogel, Preverenges (CH); Horst Matthias Pick, Preverenges (CH); Axel Kurt Preuss, New York, NY (US); Ana-Paula Tairi, Lausanne (CH); Evelyne Schmid Osborne, Sommerville Park (SG); Michael Pawlak, Laufenburg (DE)

Correspondence Address: WENDEROTH, LIND & PONACK, L.L.P., 2033 K STREET N. W., SUITE 800, WASHINGTON, DC, 20006-1021, US

(21) Appl. No.: 10/433,823

(22) PCT Filed: Dec. 4, 2001
(86) PCT No.: PCT/EP01/14134

(30) Foreign Application Priority Data
Dec. 6, 2000 (CH) 2374/00

(51) Int. Cl. G01N 33/53; G01N 33/567; C07K 14/705; C07K 16/28

(52) U.S. Cl. 435/7.2; 530/350; 530/388.22

(57) ABSTRACT

The invention relates to various embodiments of a bioanalytical reagent with at least one vesicle, generated from a living cell, comprising at least one receptor, characterized in that a mechanism of signal transduction triggered by said receptor in the cell used for vesicle generation is preserved in said vesicle, as a component of the bioanalytical reagent. The invention further relates to methods for production of the bioanalytical reagent according to the invention, to bioanalytical detection methods based on the application of said reagent, and to the use of said detection method and of the bioanalytical reagent.

The invention relates to various embodiments of a bioanalytical reagent with at least one vesicle, generated from a living cell, comprising at least one receptor, characterized in that a mechanism of signal transduction triggered by said receptor in the cell used for vesicle generation is preserved in said vesicle, as a component of the bioanalytical reagent. The invention further relates to methods for production of the bioanalytical reagent according to the invention, to bioanalytical detection methods based on the application of said reagent, and to the use of said detection method and of the bioanalytical reagent.

Living organisms can perceive an respond to a variety of external signals (light, hormones, odours, etc.). These signals are received and processed at cell surfaces (=plasma membrane receptors) by receptors which transmit the signals through the membrane and trigger a multitude of processes in the cell interior resulting in a cellular response.

Many plasma membrane receptors are important target molecules of therapeutically active substances. A medically important receptor family are G protein-coupled receptors (GPCR). They form the largest group of membrane-associated receptors and control the cellular response by means of the intermediate function of G proteins. Therefore, G proteins are an important target for drug application in the human body (Neer, E. J., Cell, 80 (1995) 249-257; Bourne, H. R., Curr. Opin. Cell Biol., 9 (1997) 134-1142; Wess, J., FASEB J, 11 (1997) 346-354).

The G_{α} sub-unit of G proteins plays a key role for the interaction of receptors with G proteins (Milligan, G., Mullaney, I., McKenzie, F. R., “Specificity of interactions of receptors and effectors with GTP-binding proteins in native membranes”, Biochem. Soc. Symp. 56, (1990) 21-34). According to the current accepted model for the activation of G proteins, this hetero-trimeric, membrane-associated protein decomposes into a free G_{α}, a subunit bound to GTP (guanine triphospate) and a free G_{βγ}-dimer. After GTP hydrolysis, Gₐ₋ (GDP) again associates with G_{βγ} (Willard F. S., Crouch M. F., Immunol. Cell Biol., 78 (2000) 387-394).

Another therapeutically important class of receptors comprises channel proteins which detect extracellular signals and convert them to cellular responses (F. M. Ashcroft: “Ion channels and disease” Academic Press, San Diego, 2000). The function of these channel proteins is to open and close ion channels.

The existence of the endoplasmic reticulum in the cell interior is of crucial importance for the functionality of the signal transduction mechanism mediated by the G-protein-coupled receptors. The endoplasmic reticulum is the most important calcium store, from which calcium ions (secondary messenger compound) are released into the cytoplasm after activation of G-protein-coupled receptors (see e.g. Muallem, S., Wilkie, T. M., “G protein-dependent Ca²⁺ signaling complexes in polarized cells”, Cell Calcium 26 (1999) 173-180).

Further calcium storage media of lesser importance are the cell nucleus and the mitochondria. The endoplasmic reticulum accumulates calcium ions by means of Ca²⁺ -ATPase as an ion pump and releases calcium via corresponding receptor ion channels, which are controlled by the messenger compounds inositol-1,4,5-triphosphate (IP3) and cyclic adenosine diphospate (cADP) (Brini, M. Carafoli, E.; “Calcium signalling: a historical account, recent developments and future perspectives”, Cell Mol. Life Sci., 57 (2000) 354-370). The basal Ca²⁺ concentration in the endoplasmic reticulum is about 500 μM and drops to about 100 μM when calcium ions stream into the cytoplasm (Yu, R., Hinkle, P. M., “Rapid turnover of calcium in the endoplasmic reticulum during signaling: studies with cameleon calcium indicators”, J. Biol. Chem., 274 (2000) 23648-23654).

An increase or decrease in the calcium concentration in a cell or a vesicle can be detected, for example, using an ion-selective indicator dye.

In spite of the great importance of these proteins, there is a lack of efficient screening assays to gain an understanding of the receptor functions and signal transduction processes at the molecular level, on the one hand, and to find and develop new therapeutic compounds (“drugs”) on the other. Traditional methods of investigation are based on assays (1) with whole cells, (2) with solubilized and purified receptors, or (3) with receptors reconstituted in artificial lipid membranes (Fernandes, P. A., Curr. Opin. Biotechnol., 9 (1998) 624-631; Zysk, J. R., Baumbach, W. R., Comb. Chem. Hight Throughput Screen., 1 (1998) 171-183).

(1) In-vivo screening methods utilize adequate living biological cells which produce and release natural or heterologous receptor proteins at the cell surface. In an assay, defined concentrations of test compounds are added to the aqueous phase in the environment of the living cells, in order to investigate whether these compounds associate with receptor proteins in the cell membrane (plasma membrane) in a specific way and, for example, stimulate or inhibit a cellular response, Typical cellular responses are changes in the intracellular ion composition (e.g. of the Na⁺ or K⁺ concentrations or of the pH), the triggering of secondary signal cascades (e.g. the release of cAMP (cyclic adenosine monophosphate) or of Ca²⁺) or changes in the activation of intracellular enzymes (e.g. of kinases, phosphatases, etc.). In these responses, some or all of the named processes may also occur simultaneously and/or coupled with each other. The nature, strength and time dependence of the intracellular responses provide important information both on the interaction between test compound and plasma membrane receptor and on the subsequent signal transduction process. In general, the binding of a test compound to the cell surface is measured using a label associated directly or indirectly with this compound, or in a competitive assay using a labeled competitor (Smith R. G., Sestili M. A., Clin. Chem., 26 (1980) 543-550; Zuck P., Lao Z., Skwish S., Glickman J. F., Yang K., Burbaum J., Inglese J., Proc. Natl. Acad. Sci. USA, 96 (1999) 11122-11127). In this case, for example, it is possible to investigate whether the binding of the test compound leads to receptor activation.

In general, an inhibition of activation is investigated in the presence of a known agonist, by studying the effect of the test compound on the activation of this agonist. Such standard methods are described, for example, in: Fernandes P. B., Curr. Opin. Chem. Biol., 2 (1998) 597-603 und in Gonzalez J. E., Negulescu P. A., Curr. Opin. Biotechnol., 9 (1998) 624-631.

Analytical methods based on the use of living cells have a number of inherent disadvantages: (I.) The laboratory infrastructure necessary for a continuous cultivation of cells is relatively complex. (II.) As a result of changes in cell physiology, living biological cells are constantly changing their properties. These changes comprise differences in the status of the cellular growth cycle, in differentiation, and in the strength of protein expression, making it difficult to establish reproducible, equivalent experimental conditions in parallel or repetitive assays. (III.) A further disadvantage is, for example, that the possibilities for miniaturizing assays based on whole cells are limited by the required volumes of nutrients (food) to be supplied.

In pharmacological drug screening, relatively time-consuming ligand binding tests and receptor functionality tests are still generally performed separately (Hodgson, J. Bio/Technology 9 (1992) 973). On the other hand, membrane proteins, such as the G-protein-coupled receptors and channel-forming receptors, are among the most important target proteins for active drugs (Knowles J, “Medicines for the new millenium hunting down diseases”, Odyssey Vol. 3 (1997)). In this context, classical patch-clamp methods are still applied as functional receptor test methods. The advantage of these electrophysiological methods is that the function of the corresponding receptors associated with channel-forming proteins can be measured directly. The method is highly specific and extremely sensitive—in principal, it is possible to measure the channel activity of individual receptors. In this case, glass micropipets with an opening diameter of typically 0.1-10 μm are positioned on the surface of a biological cell. The membrane surface that is covered by the micropipet is called a “patch”. If the contact between the glass electrode and the cell membrane surface is sufficiently electrically isolated, the ion current across the membrane patch can be measured electrically by means of microelectrodes which are positioned both in the glass pipet and in the medium on the other side of the membrane (Hamill 0. P., Marty A. et al., “Improved patch-clamp techniques for high-resolution current recording from cells and cell-free membrane patches”, Pflügers Arch., 391 (1981) 85-100).

In the context of drug screening, however, the traditional patch-clamp technique also has crucial disadvantages. Patch-clamp measurements are very time-consuming, require specially trained personnel with long experience in this field and are practically not applicable for HTS (“High Throughput Screening”).

Recently, automatic methods have been developed which allow arrays of glass microelectrodes to be positioned on suitably arranged cells or, conversely, cells or membrane fragments to be automatically (in electrical fields or by means of suitable flow devices) positioned at (sub-)micrometer-sized apertures in solid carriers, such as glass plates, silicon wafers, the surface of which may optionally be chemically modified (Vogel H., Schmidt C., “Positionierung und elektrophysiologische Charakterisierung einzelner Zellen und rekonstituierter Membransysteme auf mikrostrukturierten Trägern”, PCT/11398/01150, WO 99/31503; Schmidt C., Mayer M., Vogel H., “A chip-based biosensor for functional analysis of single ion channels”, Angew. Chemie Int. Ed., 39 (2000) 3137-3140; Klemic K. G., Buck E. et al., “Quartz microchip partitions for improved planar lipid bilayer recording of single channel currents”, Biophys. J 26-6A; Klemic K. G., et al., “Design of a microfabricated quartz electrode for ultra-low noise patch clamp recording”, Biophys. J A399).

In summary, however, it has to be concluded that the use of “native” membrane vesicles, i.e. of vesicles generated from a living cell, is described for none of these methods.

(2) Assays with receptor proteins solubilized in detergents are elaborate and generally require purification from a natural source (organism) or application of a recombinant expression system. In general, the receptor activity is only partially retained in the course of this complex procedure. In addition to receptor purification, binding assays using fluorescent or radioactive marker molecules (“labels”) have to be developed. By means of such binding assays, the nature of the interaction between an agonist or antagonist and a receptor can be investigated, but not the stimulation or inhibition of the signal transduction cascade, which is a central part of plasma membrane receptor function.

(3) Other traditional methods are based on receptors reconstituted in lipid vesicles. This requires first the isolation and purification of the receptor under consideration in suitable detergents and then the insertion of the receptor, solubilized in a detergent, into synthetic or “native” lipid bilayer vesicles. The time-consuming purification requires either receptor-rich natural cells or recombinant over-expression in cells. Reconstitution protocols have to be adapted individually for the actual receptor under consideration. Changes in the composition of the lipid phase or of the aqueous phase can have a marked impact on the efficiency of receptor insertion. The orientation of the receptor cannot be controlled during reconstitution in the vesicle membrane.

Both the binding of test compounds to the receptor and the activation of ion currents can be investigated using receptors reconstituted in vesicles. However, these vesicles do not contain the components for the complete signal transduction cascade, such as G-proteins, protein kinases, phosphatases, phosphoinosites or phospholipases, which can only hardly be reconstituted in vesicles together with receptors.

Therefore, there is a need for a bioanalytical reagent wherein receptors are provided in such a form and in such a biocompatible environment that the complete mechanism of signal transduction associated with the receptor under consideration is available for a bioanalytical investigation method and that changes in the receptor response and/or in the other components involved in the mechanism of signal transduction, can be tested in the presence of different biological or biochemical or synthetic components supplied in a sample or resulting from other changes of other external parameters influencing the transduction mechanism. It is of major importance here to avoid the mentioned disadvantages of assays based on whole living cells, i.e. the high variation of the test results due to the continuous change in living organisms and the frequent difficulties of assigning cause-and-effect relationships. This is due to the complex nature of whole cells which contain many additional biochemical components that might not be directly involved in the mechanism of signal transduction by a receptor, but which can also be effected in their function during a test procedure, which may lead to further changes in the observed test results.

According to the invention, the need for such a reagent is met by providing a bioanalytical reagent with at least one vesicle generated from a living cell comprising at least one receptor, wherein a mechanism of signal transduction triggered by said receptor in the cell used for vesicle generation is retained in said vesicle as a component of the bioanalytical reagent.

An important characteristic of many preferred embodiments of the reagent according to the invention is that a vesicle as a component of said reagent comprises besides said receptor further cell products and/or cell proteins which are involved in said mechanism of signal transduction besides said receptor, for example upon an increase or decrease in the concentration of secondary messenger compounds. The vesicle preferably comprises in additional further molecules suitable as indicators, which generate a signal or a corresponding signal change as a consequence of the change in concentration of these secondary messenger compounds, which can be monitored in a bioanalytical detection method.

In patent application WO 97/45534, a method is described for the cultivation of mammalian cells in the presence of immobilized vesicles which have been generated from a certain type of mammalian cell. In a manner similar to that described in this patent application, cytochalasin B, amongst other compounds, is used also according to the present invention for the generation of vesicles from living mammalian cells. However, in WO 97/45534 no hints are given to indicate the use of such vesicles in bioanalytical detection methods; i.e. especially not for the investigation of ligand-receptor interactions. The transduction mechanism of receptors that might optionally be associated with the vesicles and the influence of the method of cultivation thereon are not within the scope of applications described in WO 97/45534, and are consequently not discussed in any manner therein.

The generation of vesicles from living cells upon application of the fungal toxins cytochalasin B or D is known from the literature (see for example: Henson J. H., “Relationships between the actin cytoskeleton and cell volume regulation”, Microsc. Res. Tech., 47 (1999) 155-162; Brown S. S., Spudich J. A., “Mechanism of action of cytochalasin: evidence that it binds to actin filament ends”, J. Cell. Biol., 88 (1981) 487-491; Atlas S. J., Lin S., “Dihydrocytochalasin B. Biological effects and binding to 3T3 cells”, J. Cell. Biol., 76 (1978) 360-370).

This method for the preparation of so-called “native” vesicles can be applied both to adherent cells and to cells growing in suspensions. These fungal toxins act on the actin cytoskeleton. The cytoskeleton is a dynamic network responsible for various essential biological functions in the cell, such as cell division, regulation of cell volume, change of cell form and cell movement. It has been shown that cytochalasin B and D bind to the polymerization end of actin filaments and prevent their extension by inhibiting the attachment of further globular actin monomers.

After application of cytochalasin, the cells adapt a round form. Microfilaments contract and condense to local aggregates in the cell cortex. The otherwise continuous actin cytoskeleton becomes fragmentary. Supported by the cytoplasmic pressure, this effect leads to an expansion of the endoplasm in these regions. As a result, budding of the cell membrane occurs at these locations. These buds are either bullous or pedunculate in form. In some cases without further external influences, the buds are pinched off as vesicles or can be detached from the cell surface through the application of shear forces.

In a recent publication (Kask P., Palo K., Fay N., Brand L., Mets U., Ullmann D., Jungmann J., Pschorr J., Gall K., “Two-dimensional fluorescence intensity distribution analysis: theory and applications”, Biophys. J, 78 (2000) 1703-1713) the binding of fluorescent ligands to a high-affinity, human somatostatin receptor (SSTR-2) is demonstrated by fluorescence measurements using vesicles generated from cells (Schoeffter P., Perez J., Langenegger D., Schupbach E., Bobirnac I., Lubbert H., Bruns C., Hoyer D., “Characterization and distribution of somatostatin SS-1 and SRIF-1 binding sites in rat brain: identity with SSTR-2 receptors”, Eur. J. Pharmacol., 289 (1995) 163-173). For this study, membrane vesicles were generated from living cells using a glass homogenizer. In this procedure, the cells are broken open, and the signal transduction cascade, i.e. in particular further cell proteins essential for signal transduction, are most likely destroyed. In the process, cytoplasmic vesicles are also generated, besides plasma membrane-based vesicles. A later separation of these vesicles of different origin is not possible.—In the special case of the somatostatin receptor the separation of the vesicle types may not be essential for the outcome of an experiment, because somatostatin receptors are transported to the vesicle interior when they bind to agonists, and they can be localized there upon application of antibodies (Rocheville M., Lange D. C., Kumar U., Sasi R., Patel R. C., Patel Y. C., “Subtypes of the somatostatin receptor assemble as functional homo- and heterodimers”, J. Biol. Chem,. 275 (2000) 7862-7869). On the other hand, an enrichment of the fraction of vesicles from the plasma membrane is important for applications when using receptors of only very low natural abundance.

Contrary to the above methods, the controlled method for vesicle generation, according to the present invention, preserves the components of the signal transduction cascade, i.e. in particular, besides a receptor, the cell proteins involved in the signal transduction and their function also remain intact.

Said process of cell homogenization can be effected by addition of protease inhibitors. In general, the protease inhibitors block the enzymatic digestion of the membrane receptors by released cell proteases. The method for the production of the bioanalytical reagent according to the invention, upon generating vesicles using cytochalasin B, avoids the external addition of protease inhibitors. This can be advantageous, because additional components (in this case most often mixtures of protease inhibitors) in a test solution may affect the receptor-ligand interaction.

The present invention enables the adaptation of the traditional assays and detection methods for investigation of receptor-ligand interactions and of the subsequent signal transduction to a miniaturized format. According to the invention, the generated vesicles are used as compartments for receptors, wherein these compartments may additionally comprise further essential cell proteins, which may also be produced in a recombinant manner from the same cell as the vesicles to be generated.

These vesicles, as compartments derived from but independent of cells, may be frozen for storage, so that a constant quality of the sequentially used reagent aliquots of such a production lot can be assured for a lengthy period of time.

For example, the vesicles comprise either cytosolic products generated by a recombinant method (e.g. GFP, “green fluorescent protein”) or are, for example, applied as carriers for over-expressed receptors (e.g. serotonin receptor type 3, 5HT_{3A}). According to the invention, the vesicles can also serve as compartments for other recombinant products, if they are soluble in the cytosol or comprise cell surface proteins.

To meet the demand for ever more information on intracellular and extracellular biological interactions at the molecular level, there is a need for fast, parallel and highly sensitive analytical methods with minimum sample consumption.

For the determination of multiple analytes, the methods currently used in particular are those wherein the detection of different analytes is performed in discrete sample compartments or wells of so-called microtiter plates. The plates most widely used in these methods are those with an arrangement of 8×12 wells on a footprint of typically 8 cm×12 cm (see, for example, Corning Costar catalogue no. 3370, 1999), wherein a volume of several hundred microliters is required for filling a single well. For many applications, however, it would be desirable to achieve a marked reduction in the sample volume, not only to reduce the required amount of reagents and samples, which in some cases may be available in only small quantities, but also to reduce the length of diffusion paths and thus of the assay performance times in the case of assays in which biological or biochemical or synthetic recognition elements for the recognition of one or more analytes in a sample are immobilized on the wall of a sample compartment.

To reduce sample volumes and increase sample throughput, especially for screening methods, plates with an increased density (16×24 wells=384 wells and 32×48 wells 1536 wells) have been developed and commercialized, while retaining the footprint of the standard microtiter plates. This approach allows laboratory robots which are adapted to the established industrial standard to be largely retained (apart from the higher density of address points for reagent supply to the plates). Another approach was to abandon the classical plate footprint and to design the size of the wells exclusively for the sample volumes necessary for a certain application. Such arrangements have become known as “nanotiter plates”, the capacity of the individual sample compartment in some cases being no more than a few nanoliters. This technical solution, however, means dispensing with the currently widespread laboratory robots which are adapted to the classical microtiter plate standard and developing a new laboratory infrastructure optimized for the miniaturized formats. The associated additional expense is probably one of the main reasons for the fact that these miniaturized format have so far not become established on the market.

As part of such methods for the detection of analytes in one or more samples, optical methods, for example based on the determination of changes in absorption or luminescence, were increasingly developed in the past, because these methods can be performed as contactless procedures without any major repercussions on the sample. The term “luminescence” is used in this application to denote the spontaneous emission of photons in the ultraviolet to infrared spectrum after optical or nonoptical excitation, such as electrical, chemical, biochemical, or thermal excitation. For example, chemiluminescence, bioluminescence, electroluminescence and especially fluorescence and phosphorescence are included in the term “luminescence”.

The classical measurement methods, such as absorption or fluorescence measurements, are generally based on the direct illumination of a sample volume in a sample compartment or a measurement field on the inner wall of a sample compartment of a liquid sample. The disadvantage of these arrangements is that, besides the excitation volume or the excitation area wherein a signal for the detection of an analyte is to be generated, a substantial part of the environment is generally exposed to excitation light, which can lead to the disadvantageous generation of interfering background signals.

With increasing miniaturization of sample compartments, the proportion of interfering environmental light, especially as a result of reflections or luminescence from the wall surfaces of these compartments, is increased further, because the relative amount of the surface contributions to the total signal increases as the observation volume is reduced. At the same time, the achievable sample signal decreases in proportion to the sample volume.

In the past, essentially two approaches have been followed to improve the ratio between the measurement signal from optionally no more than a few analyte molecules to be detected in a sample and the interfering background signal. One of the two approaches was to restrict the observation volume to these few molecules and the other to restrict the detection to that surface on which a biological or biochemical interaction occurs.

The first of the two approaches mentioned is based on the application of confocal microscopy. One example that should be mentioned is fluorescence correlation spectroscopy (FCS), developed by Eigen and Riegler, which allowed the detection of individual molecules. Using this technique, for example, signaling proteins in individual cells have been investigated (Cluzel P., Surette M., Leibler S., “An ultrasensitive bacterial motor revealed by monitoring signaling proteins in single cell”, Science, 287 (2000) 1652-1655). Since such studies entail the determination of individual processes in discrete cells or molecules, however, numerous individual measurements are necessary for a quantitative analytical conclusion to be drawn with statistical relevance, which overall results in a long time exposure despite the high sensitivity of these methods for detecting individual molecules.

This disadvantage can be avoided using the second approach, through a spatially selective analyte detection on a macroscopic interaction surface.

Following this approach, numerous measurement arrangements have been developed, wherein the detection of an analyte is based on its interaction with the evanescent field that is associated with light guidance in an optical waveguide, wherein biochemical or biological or synthetic recognition elements for specific recognition and binding of analyte molecules are immobilized on the surface of the waveguide. When a light wave is coupled into an optical waveguide surrounded by optically rarer media, i.e. media of lower refractive index, the light wave is guided by total reflection at the interfaces of the waveguiding layer. In this arrangement, a fraction of the electromagnetic energy penetrates the media of lower refractive index. This portion is termed the evanescent (=decaying) field. The strength of the evanescent field depends to a very great extent on the thickness of the waveguiding layer itself and on the ratio of the refractive indices of the waveguiding layer and of the media surrounding it. In the case of thin waveguides, i.e. layer thicknesses that are the same as or smaller than the wavelength of the light to be guided, discrete modes of the guided light can be distinguished. As an advantage of such methods, the interaction with the analyte is limited to the penetration depth of the evanescent field into the adjacent medium, being of the order of some hundred nanometers, and interfering signals from the depth of the (bulk) medium can be largely avoided. The first proposed measurement arrangements of this type were based on highly multimodal, self-supporting single-layer waveguides, such as fibers or plates of transparent plastics or glass, with thicknesses from some hundred micrometers up to several millimeters.

For improved sensitivity and at the same time easier manufacturing in mass production, planar thin-film waveguides were used in the years that followed. In the simplest case, a planar thin-film waveguide consists of a three-layer system: support material (substrate), waveguiding layer, superstrate (i.e. the sample to be analyzed), wherein the waveguiding layer has the highest refractive index. Additional intermediate layers can further improve the action of the planar waveguide.

Several methods for the incoupling of excitation light into a planar waveguide are known. The earliest methods used were based on butt coupling or prism coupling, wherein generally a liquid is introduced between the prism and the waveguide in order to reduce reflections resulting from air gaps. These two methods are particularly suitable with respect to waveguides of relatively large layer thickness, i.e. especially self-supporting waveguides, and with respect to waveguides with a refractive index significantly below 2. For incoupling of excitation light into very thin waveguiding layers of high refractive index, however, the use of coupling gratings is a significantly more elegant method.

Various methods of analyte determination in the evanescent field of lightwaves guided in optical film waveguides can be distinguished. Based on the measurement principle applied, for example, a distinction can be drawn between fluorescence, or more general luminescence methods on the one hand and refractive methods on the other. In this context, methods for the generation of surface plasmon renonance in a thin metal layer on a dielectric layer of lower refractive index can be included in the group of refractive methods, provided the resonance angle of the launched excitation light for generation of the surface plasmon resonance is taken as the quantity to be measured. Surface plasmon resonance can also be used for the amplification of a luminescence or the improvement of the signal-to-background ratios in a luminescence measurement. The conditions for generation of a surface plasmon resonance and the combination with luminescence measurements, as well as with waveguiding structures, are described in the literature, for example in U.S. Pat. No. 5,478,755, No. 5,841,143, No. 5,006,716, and No. 4,649,280.

In the case of the refractive measurement methods, the change in the effective refractive index resulting from molecular adsorption to or desorption from the waveguide is used for analyte detection. This change in the effective refractive index is determined, in the case of grating coupler sensors, from changes in the coupling angle for the in- or out-coupling of light into or out of the grating coupler sensor and, in the case of interferometric sensors, from changes in the phase difference between measurement light guided in a sensing branch and a referencing branch of the interferometer.

The aforesaid refractive methods have the advantage that they can be applied without using additional marker molecules, so-called molecular labels. The disadvantage of these label-free methods, however, is that, in view of the low selectivity of the measurement principle, the detection limits achievable with these methods are confined to pico- to nanomolar concentration ranges, depending on the molecular weight of the analyte, which is not sufficient for many applications of modern trace analysis, for example for diagnostic applications.

For achieving lower detection limits, luminescence-based methods appear more suitable in view of the higher selectivity of signal generation. In this arrangement, luminescence excitation is confined to the penetration depth of the evanescent field into the medium of lower refractive index, i.e to the immediate proximity of the waveguiding area, with a penetration depth of the order of some hundred nanometers into the medium. This principle is called evanescent luminescence excitation.

By means of highly refractive thin-film waveguides, based on a waveguiding film measuring only a few hundred nanometers in thickness on a transparent support material, it has been possible to increase sensitivity considerably in recent years. In WO 95/33197, for example, a method is described, wherein the excitation light is coupled into the waveguiding film via a relief grating as diffractive optical element. The isotropically emitted luminescence from substances capable of luminescence, which are located within the penetration depth of the evanescent field, is measured by suitable measurement devices, such as photodiodes, photomultipliers or CCD cameras. The portion of evanescently excited radiation that has back-coupled into the waveguide can also be out-coupled via a diffractive optical element, such as a grating, and be measured. This method is described, for example, in WO 95/33198. The in-coupling and out-coupling grating in this method may also be identical, because each in-coupling grating can be used as an out-coupling grating under the same conditions as for in-coupling, in view of the reversibility of the light path.

For the simultaneous or sequential performance of exclusively luminescence-based, multiple measurements with essentially monomodal, planar inorganic waveguides, for example in the specification WO 96/35940, arrangements (arrays) have been proposed wherein at least two discrete waveguiding areas are provided on one sensor platform, such that the excitation light guided in one waveguiding area is separated from other waveguiding areas. By means of such an arrangement it is possible, in particular, to determine different analytes simultaneously in an applied sample, using different recognition elements immobilized in discrete measurement areas (d).

According to the present invention, spatially separated measurement areas (d) should be defined by the area that is occupied by biological or biochemical or synthetic recognition elements immobilized thereon for recognition of an analyte in a liquid sample. These areas may have any geometry, for example the form of dots, circles, rectangles, triangles, ellipses or lines.

For the investigation of receptor-ligand interactions, especially the functionality of a transduction mechanism controlled by a receptor, there is a need for a solid carrier, in particular for a sensor platform with high detection sensitivity, designed in such a way that the mechanism of signal transduction, is not impaired, in particular by the immobilization of the receptor on a solid surface. Various embodiments of solid carriers and/or sensor platforms are provided within the scope of this invention.

A first subject of the invention is a bioanalytical reagent with at least one vesicle, generated from a living cell, comprising at least one receptor, characterized in that a mechanism of signal transduction triggered by said receptor in the cell used for vesicle generation is preserved in said vesicle as a component of the bioanalytical reagent.

The receptor associated with said vesicle may be located both inside the vesicle and on the vesicle membrane. It is preferred if the receptor is integrated into the vesicle membrane.

Said mechanism of signal transduction may be triggered in this case by the effect both of external signals or of signals inside the vesicle or of signal-generating biological or biochemical or synthetic components possibly added externally. “External or vesicle-internal signals” are here understood, for example, to be changes in macroscopic properties, such as changes in ion concentrations in the medium or in the vesicle. By contrast, “signal-generating biological or biochemical or synthetic components” are understood, for example, to be ligands binding specifically to a receptor.

An important characteristics of numerous preferred embodiments of the reagent according to the invention is that a vesicle as a component of said reagent comprises further cell products and/or cell proteins, besides said one or more receptors, which are involved in said mechanism of signal transduction, besides said one or more receptors, for example upon an increase or a decrease of secondary messenger compounds within the vesicle.

Said one or more vesicles as a component of the bioanalytical reagent according to the invention may be generated from a eukaryotic cell or from a cell of native tissue.

It is preferred that the interior of said one or more vesicles is free from cell nucleus material, (chromosomal DNA), so that replication processes do not occur within said one or more vesicles. This is an important aspect for applications with “native” vesicles, which are free of heterologous DNA (for example upon the insertion of vesicles as a carrier into another target organism).

For many applications of the bioanalytical reagent according to the invention, it is preferred that said one or more vesicles have a diameter of 50 nm-5000 nm. A diameter of 100 m-2000 nm is especially preferred.

A characteristic of many embodiments of the bioanalytical reagent according to the invention is that said one or more receptors are present in natural form in said one or more vesicles as a component of the bioanalytical reagent.

For other applications it is preferred that said one or more receptors are present in a modified form in said one or more vesicles as a component of the bioanalytical reagent. For example, a receptor may be present as a fusion protein, for example by fusion with a fluorescent protein such as GFP (green fluorescent protein, Tsien R. Y., “The green fluorescent protein”, Annu. Rev. Biochem. 67 (1998) 509-544) or BFP (blue fluorescent protein) or RFP (red fluorescent protein).

It is characteristic of many embodiments that said one or more receptors are present in recombinant form in said one or more vesicles as a component of the bioanalytical reagent.

An important characteristic of the bioanalytical reagent according to the invention is that a binding capability of said one or more receptors to a specific ligand is preserved, this binding capacity being present in said vesicle-generating cell and the receptor being associated with the vesicle as a component of the bioanalytical reagent.

The one or more receptors may be selected from the group of signal-transducing receptors that is formed by plasma membrane receptors, such as ion channel receptors, G-protein-coupled receptors (GPCR), orphan receptors, enzyme-coupled receptors, such as receptors with an intrinsic tyrosine kinase activity, receptors with an intrinsic serine/threonine kinase activity, furtheron by receptors for growth factors (peptide hormone receptors), receptors for chemotactic substances, such as the class of chemokine receptors, and by intracellular hormone receptors, such as steroid hormone receptors.

Characteristic of some embodiments of the bioanalytical reagent according to the invention is that said one or more receptors are in contact with the outer vesicle membrane. It is then preferred that, with respect to the surface of the outer vesicle membrane, the areal density of receptors that are in contact with the outer vesicle membrane is of similar order of magnitude or greater than the corresponding density of these receptors in the vesicle-generating living cell.

Characteristic of other embodiments of the bioanalytical reagent according to the invention, by contrast, is that said one or more receptors are located in the interior of the vesicle. In this case, it is preferred that, with respect to the vesicle volume, the volume density of receptors located in the interior of a vesicle is of similar order of magnitude or greater than the density of the receptors in the vesicle-generating living cell.

It is of course particularly advantageous if, in the sense of an enrichment of receptors and/or of their ligand binding sites, the process of production of the vesicle from a living cell allows said areal density of the receptors in the vesicle membrane in the case of receptors being in contact with the outer vesicle membrane, or their volume density inside the vesicle in the case of ligand binding sites located in the vesicle interior, to be increased with respect to the corresponding densities in the original cell.

It is preferred that said one or more vesicles comprise, besides said one or more receptors, further biological compounds (components) from the group that is formed e.g. by G proteins and G-protein regulators (e.g. rasGAP), enzymes such as adenylate cyclases, phospholipases which form intracellular secondary messenger compounds (e.g. cAMP (cyclic adenosine monophosphate), cGMP (cyclic guanosine monophosphate), diacyl glycerol (DAG) or inositol triphosphate (IP3)), enzymes such as serine, threonine and tyrosine kinases, and tyrosine phosphatases that activate or inhibit proteins by phosphorylation or de-phosphorylation.

The bioanalytical reagent according to the invention is also suitable for application in a living organism, for example, to perform bioanalytical studies in the organism at a specific, pre-determined site, for example by means of indicator compounds incorporated in the vesicle. For example it is advantageous for such applications if biological, biochemical or synthetic compounds, such as cell surface proteins or cell surface sugars, are associated with the outer membrane of the one or more vesicles, these compounds being used for the transport of said vesicle to pre-determined destinations, such as cells and/or organs and/or pre-determined tissue in a living organism, and/or for the binding to a biological or biochemical or synthetic recognition element which specifically recognizes and binds said biological or biochemical or synthetic recognition element.

In order to reduce nonspecific binding of a vesicle, as a component of a bioanalytical reagent according to the invention, to a surface brought into contact with the vesicle, it may be advantageous if lipids comprising, for example, hydrophilic polymers (such as polyethylene glycols) are additionally integrated into the vesicle membrane after generation of said vesicle from a living cell. Vesicles with surface-associated polymers are described, for example, in the international application PCT/EP 00/04491.

Said mechanism of signal transduction, which is preserved in the bioanalytical reagent according to the invention, may comprise a mechanism from among the group of mechanisms that is formed e.g. from ion conducting, G-protein coupling, activation or inhibition of intra-vesicular ion channels, intra-vesicular release of calcium, protein activation or inhibition by enzymatic phosphorylation or de-phosphorylation (kinase cascades; phosphatases), and release or enzymatic formation of secondary messenger compounds, such as cAMP, cGMP or diacyl glycerol (DAG), inositol triphosphate (IP3).

Said mechanism of signal transduction may comprise a (secondary) functional response of the one or more vesicle-associated receptors after a primary specific interaction of said receptor with one or more natural and/or synthetic ligands contained in a sample that is brought into contact with said vesicle.

The mechanism of signal transduction may also comprise the activation of an ion channel of a receptor associated with a vesicle, as a component of said bioanalytical reagent.

It is also possible that said mechanism of signal transduction comprises the binding of a G-protein to a receptor associated with a vesicle, as a component of said bioanalytical reagent.

Said mechanism of signal transduction may also comprise the internal release of ions, such as Ca²⁺, or of other messenger compounds, such as cAMP or cGMP.

The mechanism of signal transduction may also comprise the enzymatic decomposition of a substrate by a vesicle-associated enzyme to form a product. In this case, said vesicle may be located in the vesicle interior or may be associated with the vesicle membrane.

Characteristic of a specific embodiment of the bioanalytical reagent according to the invention is that a (secondary) functional response as part of said mechanism of signal transduction occurs after interaction between one or more natural and/or synthetic ligands or co-factors contained in a sample brought into contact with said vesicle on the one hand and naturally or recombinantly generated proteins associated with said vesicle on the other.

It is preferred that said one or more vesicles additionally comprise components for generation of an experimentally detectable signal.

These additional components for generation of an experimentally detectable signal may be associated with further biological compounds (components) which are associated in turn with the one or more vesicles as a component of said bioanalytical reagent.

In this case, said further biological compounds (components) may originate from the group that is formed e.g. by G proteins and G-protein regulators (e.g. rasGAP), enzymes such as adenylate cyclases, phospholipases which form intracellular secondary messenger compounds (e.g. cAMP (cyclic adenosine monophosphate), cGMP (cyclic guanosine monophosphate), diacyl glycerol (DAG) or inositol triphosphate (IP3)), enzymes such as serine, threonine and tyrosine kinases, and tyrosine phosphatases that activate or inhibit proteins by phosphorylation or de-phosphorylation.

The said additional components for generation of an experimentally detectable signal may also be associated with a receptor or may be parts of fusion proteins associated with the vesicle.

Said additional components for generation of an experimentally detectable signal may be selected from the group of components formed by absorptive indicators and luminescent indicators, luminescence labels, luminescent nanoparticles, absorptive indicator proteins and luminescent indicator proteins, such as BFP (“blue fluorescent protein”), GFP (“green fluorescent protein”) or RFP (“red fluorescent protein”), artificial luminescent (i.e. in particular fluorescent) amino acids, radioactive labels, spin labels, such as NMR labels or ESR labels, ion indicators, especially pH and calcium indicators, or potential-dependent indicators, such as potential-dependent luminescence labels, or redox complexes.

Characteristic of a preferred embodiment of the bioanalytical reagent according to the invention is that said additional components for generation of an experimentally detectable signal are generated from the same cell from which the vesicle was generated.

Characteristic of another possible embodiment is that said additional components for generation of an experimentally detectable signal are inserted into the cell from which the vesicle is generated before production of the vesicle.

However, said additional components for generation of an experimentally detectable signal may also be inserted into the vesicle after its formation.

A very important and advantageous characteristic for the application of the bioanalytical reagent according to the invention in practice is that the functionality of a receptor associated with a vesicle as a component of said bioanalytical reagent is preserved upon storage under deep-frozen conditions for at least one week, preferably for at least one month, especially preferably for at least one year. Precise conditions for the deep-freezing of vesicles as part of a bioanalytical reagent according to the invention are described in Example 3. In this case, the “preservation of the functionality” of said receptor is intended to mean that a mechanism of signal transduction to be triggered by said receptor in the vesicle is also when the vesicle is thawed out again intact after storage of the vesicle under the conditions described.

Exposed to different conditions, for example under sterile conditions in cooled buffer solution, i.e. at a temperature below ambient temperature, e.g. at 4° C., a bioanalytical reagent according to the invention is characterized by a shelf life of at least one week.

A further subject of the present invention is a method for production of a bioanalytical reagent with a vesicle generated from a living cell according to any of the embodiments mentioned above, wherein said vesicle was produced from a living cell comprising at least one receptor, and wherein a mechanism of signal transduction triggered by said receptor in said living cell is preserved in said vesicle as a component of the bioanalytical reagent.

It is preferred if the constriction and pinching off of said vesicle from said living cell is effected after application of cytochalasin B and/or cytochalasin D.

It is also preferred if the method according to the invention is performed without application of protease inhibitors. In the case of other methods wherein cell proteases are released, there is the risk that a certain decomposition of receptor proteins may occur despite the addition of a protease inhibitor or mixtures of protease inhibitors. Protease inhibitors also have to be removed in additional purification steps.

The method according to the invention may comprise the application of shear forces and/or of centrifugation steps, for example upon exposure to a gradient of sucrose, and/or the application of chromatographic steps, for example by separation into fractions of different size distributions, and/or the application of filtration steps and/or the application of electrophoretic methods.

By means of the method according to the invention, said one or more vesicles may be generated from a eukaryotic cell. Said one or more vesicles may also be generated from a cell of native tissue.

Characteristic of an important embodiment of the method according to the invention is that the interior of a vesicle produced by said method is free of cell nucleus material, so that replicative processes do not occur.

For a preferred embodiment of the method according to the invention, it is characteristic that a vesicle produced by said method has a diameter of 50 nm-5 000 nm, especially preferably of 100-2 000 nm.

For many applications, it is preferred if a receptor in a vesicle produced by this method, as a part of the bioanalytical reagent, is provided in natural form.

For other applications it is preferred that a receptor in a vesicle produced by this method, as a part of the bioanalytical reagent, is provided in a modified form. Said receptor, may be, for example, provided as a fusion protein, e.g. by fusion of a fluorescent protein such as GFP (green fluorescent protein) or BFP (blue fluorescent protein) or RFP (red fluorescent protein). Another example is the fusion of a GPCR and a G-protein.

Often it is also advantageous if a receptor in a vesicle produced by this method is provided in recombinant form.

An important characteristic of the method according to the invention comprises the preservation of a binding capability of said one or more receptors to a specific ligand, this binding capability being present in said vesicle-generating cell and the receptor being associated with the vesicle as a component of the bioanalytical reagent.

The one or more receptors may be selected from the group of signal-transducing receptors that is formed by plasma membrane receptors, such as ion channel receptors, G protein-coupled receptors (GPCR), orphan receptors, enzyme-coupled receptors, such as receptors with an intrinsic tyrosine kinase activity, receptors with an intrinsic serine/threonine kinase activity, furtheron by receptors for growth factors (peptide hormone receptors), receptors for chemotactic substances, such as the class of chemokine receptors, and by intracellular hormone receptors, such as steroid hormone receptors.

Preferred are embodiments of the method according to the invention wherein, with respect to the surface of the outer vesicle membrane, the areal density of receptors that are in contact with the outer vesicle membrane is of a similar order of magnitude or greater than the corresponding density of these receptors in the vesicle-generating living cell.

Also preferred are embodiments of the method wherein, with respect to the vesicle volume, the volume density of receptors located in the interior of a vesicle is of similar order of magnitude or greater than the density of these receptors in the vesicle-generating living cell.

Characteristic of further embodiments of the method according to the invention is that said one or more vesicles produced by this method comprise, besides said one or more receptors, further biological compounds (components) from the group that is formed e.g. by G proteins and G-protein regulators (e.g. rasGAP), enzymes such as adenylate cyclases, phospholipases which form intracellular secondary messenger compounds (e.g. cAMP (cyclic adenosine monophosphate), cGMP (cyclic guanosine monophosphate), diacyl glycerol (DAG) or inositol triphosphate (IP3)), enzymes such as serine, threonine and tyrosine kinases, and tyrosine phosphatases that activate or inhibit proteins by phosphorylation or de-phosphorylation.

Characteristic of further embodiments of the method is that biological, biochemical or synthetic compounds, such as cell surface proteins or cell surface sugars, are associated with the outer membrane of the one or more vesicles produced by this method, these compounds being used for the transport of said vesicle to pre-determined destinations, such as cells and/or organs and/or pre-determined tissue in a living organism, and/or for the binding to a biological or biochemical or synthetic recognition element, which specifically recognizes and binds said biological or biochemical or synthetic recognition element.

To reduce nonspecific binding of a vesicle, as part of a bioanalytical reagent according to the invention, to a surface that is to be brought into contact with the vesicle, it may be advantageous if, after production of said vesicle from a living cell, lipids comprising for example hydrophilic polymers (such as polyethylene glycols) are additionally integrated into the vesicle membrane. Vesicles with surface-associated polymers are described, for example, in PCT/EP/00/04491.

Of high importance are also embodiments of the method according to the invention wherein said one or more vesicles produced by this method additionally comprise components for generation of an experimentally detectable signal.

These additional components for generation of an experimentally detectable signal may be comprised in the further biological compounds (components) associated with the one or more vesicles as part of said bioanalytical reagent. In this case, said further biological compounds (components) may be comprised in the group that is formed e.g. by G proteins and G-protein regulators (e.g. rasGAP), enzymes such as adenylate cyclases, phospholipases which form intracellular secondary messenger compounds (e.g. cAMP (cyclic adenosine monophosphate), cGMP (cyclic guanosine monophosphate), diacyl glycerol (DAG) or inositol triphosphate (IP3)), enzymes such as serine, threonine and tyrosine kinases, and tyrosine phosphatases that activate or inhibit proteins by phosphorylation or de-phosphorylation.

Said additional components for generation of an experimentally detectable signal my also be associated with a receptor or may be parts of fusion proteins associated with the vesicle.

Said additional components for generation of an experimentally detectable signal may be selected from the group of components formed by absorptive indicators and luminescent indicators, luminescence labels, luminescent nanoparticles, absorptive indicator proteins and luminescent indicator proteins, such as BFP (“blue fluorescent protein”), GFP (“green fluorescent protein”) or RFP (“red fluorescent protein”), artificial luminescent (i.e. in particular fluorescent) amino acids, radioactive labels, spin labels, such as NMR labels or ESR labels, ion indicators, especially pH and calcium indicators, or potential-dependent indicators, such as potential-dependent luminescence labels, or redox complexes.

According to the method according to the invention, said additional components for generation of an experimentally detectable signal may be generated from the same cell from which the vesicle was produced.

The cell may also be loaded with said additional components for generation of an experimentally detectable signal before production of the vesicle.

However, said additional components for generation of an experimentally detectable signal may also be inserted into the vesicle after its formation.

A further subject of the invention is a bioanalytical detection method with a bioanalytical reagent according to any of the aforementioned embodiments, wherein said detection method is selected from the group that is formed, for example, by optical detection methods, such as refractrometric methods, surface plasmon resonance, optical absorption measurements (e.g. internal reflection methods using a highly refractive material, in combination with infrared spectroscopic measurements) or luminescence detection (e.g. fluorescence correlation spectroscopy), detection of energy or charge transfer, mass spectroscopy, electrical or electrochemical detection methods, such as electrophysiology, patch clamp techniques, impedance measurements, electronic resonance measurements, such as electron spin resonance or nuclear spin resonance, gravimetric methods (e.g. electrical crystal balance measurements), radioactive methods, or by electrophoretic measurements.

Characteristic of some of the possible embodiments of the bioanalytical detection method according to the invention is that said method is performed in homogeneous solution.

A special group of embodiments of the bioanalytical detection method according to the invention with a bioanalytical reagent according to the invention is related to special patch-clamp methods which can be performed in a single-device arrangement or in a multiple-device arrangement. Characteristic of a bioanalytical detection method according to this embodiment is that said method is performed using a measurement arrangement with at least 2 electrodes and separate compartments suitable for receiving liquids, wherein a solid carrier (preferably as an electrically isolating separation wall), comprising at least one aperture and separating at least 2 compartments, is located between two electrodes facing each other, the electrodes being of any geometrical form and each extending into at least one compartment or being in contact with at least one compartment.

Said carrier is provided as a separation wall comprising an electrically isolating material located between the electrodes. As mentioned above, the carrier is provided with an aperture and with a surface on which vesicles from a bioanalytical reagent according to the invention can be fixed. The carrier must not necessarily consist of a single piece, but it may e.g. comprise a holder to which the material which is actually relevant for membrane binding and membrane positioning may be attached or in which this material may be inserted, said material comprising at least one aperture for the binding and positioning of the membranes. Additionally, the aperture may be surrounded by a circular, tapering elevation (typically with a height in the sub-micrometer range and the aperture located in the center). Thus, a micropipet-like aperture may be generated on an otherwise essentially planar carrier.

In the presence of a potential difference over the measurement arrangement and mediated by the two or more electrodes, such a specific arrangement allows an inhomogeneous electrical field to be generated around the aperture, said field having an increasing value with decreasing distance from the aperture and said field being capable of moving vesicles electrophoretically towards the aperture, said vesicles being located close to said aperture and generated from a living cell, from a bioanalytical reagent according to the invention.

Such an arrangement also allows such vesicles to be positioned over or within the aperture by means of a hydrodynamic or electrokinetic flow or by other mechanical manipulation (e.g. by means of optical tweezers, force microscope or by a micro manipulator).

The fixation of the membranes may for example be based on electrostatic interactions between e.g. a negatively charged membrane surface and a positively charged carrier surface. If the carrier surface by itself is not provided with the desired charge, it may be modified accordingly.

It is preferred if said measurement arrangement is provided with means on one side or on both sides of the carrier which enable a supply of liquid and/or a storage of liquid and/or an exchange of liquid and/or the addition of vesicles generated from a living cell, from a bioanalytical reagent according to the invention, between carrier and electrode.

It is also preferred if the one or more apertures of said measurement arrangement have such a diameter that, in the presence of a potential difference and mediated by the two or more electrodes, an inhomogeneous electrical field is generated around the aperture, said field having an increasing value with decreasing distance from the aperture and said field being capable of moving vesicles electrophoretically towards the aperture, said vesicles being located close to said aperture and generated from a living cell, from a bioanalytical reagent according to the invention.

It is also advantageous if the one or more apertures of said measurement arrangement have such a diameter that vesicles generated from a living cell, from a bioanalytical reagent according to the invention, can be positioned over or within the aperture by means of a hydrodynamic or electrokinetic flow or by other mechanical manipulation (e.g. by means of optical tweezers, force microscope or by a micro manipulator).

Characteristic of many embodiments of this special group of bioanalytical detection methods according to the invention is that the carrier of said measurement arrangement is provided with an electrically charged surface which exerts attractive force on vesicles generated from a living cell, from a bioanalytical reagent according to the invention, or is provided with an adhesion-promoting layer for binding said vesicles on its surface.

The bioanalytical detection method according to the invention may be performed by inserting vesicles generated from a living cell, from a bioanalytical reagent according to the invention, between separation wall or carrier and electrode into a compartment filled or not filled with buffer beforehand, and by moving said vesicles towards the aperture by means of an electrical potential difference applied to the electrodes, and/or by positioning said vesicles on the aperture by hydrodynamic or electrokinetic flow and/or by positioning the vesicles on the aperture mechanically (e.g. by means of optical tweezers, force microscope or by a micro manipulator).

In particular it is possible that vesicles generated from a living cell, from a bioanalytical reagent according to the invention, are positioned on said aperture, the vesicle membranes form an electrically close contact with the carrier over the aperture, and a measurement of the (electrical) membrane resistance is enabled. During this procedure, the vesicles may preserve their form or may merge with the surface of the carrier and thus form an aperture-spanning planar membrane. In this case, a good signal-to-noise discrimination can be achieved by means of the method according to the invention.

The method also allows artificial lipid vesicles with a diameter larger than the diameter of said aperture to be added to at least one compartment, in order to generate a planar lipid bilayer on the surface of the carrier and extending over the aperture, and vesicles generated from a living cell, from a bioanalytical reagent according to the invention, then to be added to said compartment, in order to fuse said vesicles with the generated lipid membrane and to render receptors that are associated with said vesicles generated from living cells accessible for electrical or optical measurements.

Moreover, the method enables membrane proteins to be inserted into a vesicle generated from a living cell, after positioning said vesicle on an aperture.

It is preferred if a vesicle generated from a living cell located over an aperture or a planar membrane generated from said vesicle and spanning an aperture is accessible for optical measurements, especially for fluorescence measurements, or for simultaneous optical and electrical measurements, to which it is subjected.

Characteristic of a special variant of this method according to the invention based on patch-clamp techniques is that a measurement arrangement or a measurement system with several apertures on one carrier is used and that measurements on at least two apertures are performed sequentially and/or in parallel.

In particular, numerous vesicles generated from living cells, from a bioanalytical reagent according to the invention, may be arranged in an array on a solid, electrically isolating carrier, wherein said array of vesicles is brought into electrically isolating contact with an array of patch-clamp pipets in a geometrical arrangement similar to that of the vesicle array, in order to enable a simultaneous performance of electrical measurements independently of each other or simultaneous electrical and optical measurements on a large number of individual vesicles.

Further embodiments of such measurement arrangements, especially with a “patch-clamp array” and analytical detection methods based on the use thereof, which are suitable for a bioanalytical detection method according to the invention using a bioanalytical reagent according to the invention, are described in WO 99/31503. The use of these measurement arrangements and detection methods, in combination with a bioanalytical detection method according to the invention using a bioanalytical reagent according to the invention, is also a subject of the present invention.

Characteristic of numerous possible embodiments of a bioanalytical detection method according to the invention is that the one or more vesicles generated from a living cell, comprising at least one receptor, from a bioanalytical reagent according to the invention, is immobilized on the surface of a solid support.

It is advantageous if a vesicle generated from a living cell, from a bioanalytical reagent according to the invention, is also accessible in particular for mass-spectrometric investigations after immobilization on a solid support.

Characteristic of said embodiments of a bioanalytical detection method according to the invention is that a mechanism of signal transduction triggered by said receptor in said living cell is preserved in a vesicle generated from the cell after immobilization of the vesicle.

A particular subject of the invention is therefore a bioanalytical detection method with at least one vesicle immobilized on the surface of a solid support, the vesicle being generated from a living cell, comprising at least one receptor, from a bioanalytical reagent according to the invention, wherein a mechanism of signal transduction triggered by said receptor in said living cell is preserved in a vesicle generated from the cell after immobilization of the vesicle.

It is preferred if vesicles, each comprising at least one receptor, are immobilized in discrete measurement areas (d) with one or more vesicles each on the surface of said solid support.

It is further preferred if vesicles with at least two different kinds of receptor are immobilized in numerous measurement areas (d), wherein each vesicle is preferably immobilized with the same kind of receptor within an individual measurement area.

The one or more vesicles generated from a living cell may be immobilized on the surface of said solid support for example by means of covalent binding or physical adsorption (electrostatic or van der Waals interaction or hydrophilic or hydrophobic interaction or a combination of these interactions).

It is preferred if an adhesion-promoting layer is deposited between the surface of said solid support and the one or more vesicles immobilized thereon. In this case, according to the invention, the adhesion-promoting layer is designed in such a way that a mechanism of signal transduction triggered by the one or more receptors in said living cell is preserved also after immobilization of the vesicles generated from a living cell as part of a bioanalytical reagent comprising at least one receptor, according to the invention, on said adhesion-promoting layer.

It is preferred if the adhesion-promoting layer has a thickness of preferably less than 200 nm, most preferably of less than 20 nm.

The adhesion-promoting layer may comprise a chemical compound of the group of silanes, epoxides, functionalized, charged or polar polymers and “self-organized functionalized mono or multiple layers”.

Characteristic of a preferred embodiment is that the adhesion-promoting layer comprises a monomolecular layer of mainly one kind of protein, such as serum albumins or streptavidin, or of modified proteins, such as biotinylated serum albumin.

Characteristic of another preferred embodiment is that the adhesion-promoting layer comprises self-organized alkane-terminated monolayers of mainly one kind of chemical or biochemical molecules.

Especially preferred is an embodiment wherein the adhesion-promoting layer is provided as a double layer (bilayer), comprising an initial self-organized alkane-terminated anchoring layer and a second layer formed by self-organization (self-assembly) of synthetic or natural lipids.

The immobilization of the one or more vesicles generated from a living cell on the adhesion-promoting layer may be performed, for example, upon covalent binding or upon physical adsorption (electrostatic or van der Waals interaction or hydrophilic or hydrophobic interaction or a combination of these interactions).

A special embodiment of the bioanalytical detection method according to the invention, using a particularly specific variant of vesicle immobilization, comprises association with the adhesion-promoting layer of biological or biochemical or synthetic recognition elements which recognize and bind a vesicle generated from a living cell with surface-associated biological or biochemical or synthetic components for specific recognition and binding, as part of the corresponding above-described specific embodiment of a bioanalytical reagent according to the invention. These specific interactions for the recognition and binding of the vesicles to their recognition elements on the adhesion-promoting layer may for example be based on interactions with biotin/streptavidin, so-called “histidine tags” (references: Schmid E. L., Keller T. A., Dienes Z., Vogel H., “Reversible oriented surface immobilization of functional proteins on oxide surfaces”, Anal Chem 69 (1997) 1979-1985; Sigal G. B., Bamdad C., Barberis A., Strominger J., Whitesides G. M., “A self-assembled monolayer for the binding and study of histidine-tagged proteins by surface plasmon resonance”, Anal Chem 68 (1996) 490-497), sugars or peptide affinity interactions, wherein any one of the two binding partners in each case may be associated with the vesicle surface and the other anchored on the surface of said adhesion-promoting layer.

Characteristic of another embodiment of the bioanalytical detection method according to the invention is that at least one ligand for a receptor, which is bound to a vesicle generated from a living cell, from a bioanalytical reagent according to the invention, is immobilized, optionally by means of a spacer molecule, on the surface of the solid support.

In this case it is preferred if at least two different ligands for receptors, which are bound to a vesicle generated from a living cell, from a bioanalytical reagent according to the invention, are immobilized in numerous measurement areas (d), wherein preferably the same kind of ligand is immobilized within an individual measurement area.

Said ligands may be immobilized on the surface of the solid support by means of covalent binding or physical adsorption (e.g. electrostatic or van der Waals interaction or hydrophilic or hydrophobic interaction or a combination of these interactions).

It is preferred if an adhesion-promoting layer is applied between the surface of the solid support and said ligands immobilized thereon.

For the selection of the adhesion-promoting layer for immobilization of said ligands, the same preferences apply as mentioned above for an adhesion-promoting layer for immobilization of vesicles generated from a living cell, from a bioanalytical reagent according to the invention.

Characteristic of a particularly preferred embodiment of a bioanalytical detection method according to the invention is that regions between the laterally separated measurement areas, with vesicles generated from living cells (from a bioanalytical reagent according to any of the described embodiments) immobilized therein, or with ligands for receptors that are bound to vesicles generated from living cells (from a bioanalytical reagent according to any of the described embodiments) immobilized therein, and/or that regions within these measurement areas, between the compounds immobilized therein, are “passivated” in order to minimize nonspecific binding of analytes or of their detection reagents, i.e., that compounds which are “chemically neutral” towards the analyte are deposited between the laterally separated measurement areas (d) and/or within these measurement areas (d) between said immobilized compounds, the “chemically neutral” compounds preferably being composed of the groups that are formed by albumins, casein, detergents, such as Tween 20, detergent/lipid mixtures (of synthetic and/or natural lipids), synthetic and natural lipids or also hydrophilic polymers, such as polyethylene glycols or dextrans.

It is also possible to passivate an activated surface (activated for immobilization of the biological, biochemical recognition elements, the activated surface comprising e.g. poly-L-lysin or functionalized silanes comprising e.g. aldehyde or epoxy groups), for example by the addition of reducing reagents such as sodium borate (in the case of aldehyde or epoxy groups).

The material of the surface of the solid support (carrier) with immobilized vesicles generated from living cells (from a bioanalytical reagent according to the invention and any of the described embodiments) or with immobilized ligands for receptors that are bound to vesicles generated from living cells (from a bioanalytical reagent according to the invention and any of the described embodiments) may comprise a material of the group which is formed e.g. by moldable, sprayable or millable plastics, carbon compounds, metals, such as gold, silver, copper, metal oxides or silicates, such as glass, quartz or ceramics, or silicon or germanium or ZnSe or a mixture of these materials.

In this case, said solid carrier (support) may be provided in various embodiments. It may be provided e.g. as a glass or microscope plate. It may also be a microtiter plate of the type for example that is in widespread use for screening assays (for testing numerous compounds, e.g. using classical fluorescence methods or fluorescence correlation spectroscopy).

It is preferred if the surface of said solid support (carrier) is essentially planar.

Characteristic of a preferred group of embodiments of the bioanalytical detection method is said solid support (carrier) is an optical or electronic sensor platform.

In this case, it is preferred that said solid support (carrier) is transparent at least in a region of wavelengths in the ultraviolet to infrared spectrum and comprises preferably a material from the group that is formed e.g. by moldable, sprayable or millable plastics, carbon compounds, metals, metal oxides or silicates, such as glass, quartz or ceramics, or silicon or germanium or ZnSe or a mixture of these materials.

It is preferred here if said solid support is an optical waveguide used as a sensor platform. Specially preferred is an embodiment of the detection method wherein said solid support is an optical thin-film waveguide used as a sensor platform, with an initial optically transparent layer (a) with refractive index n₁ on a second optically transparent layer (b) with refractive index n₂, wherein n₁>n₂.

For example for the simultaneous analysis of multiple samples and/or for the determination of multiple analytes in one or more samples it is advantageous, if the sensor platform as a solid support is divided into two or more discrete waveguiding regions.

The material of the second optically transparent layer (b) of the sensor platform as a solid support may be selected from the group that is formed by silicates, such as glass or quartz, or transparent moldable, sprayable or millable, especially thermoplastic plastics, such as polycarbonates, polyimides, polymethyl methacrylates, or polystyrenes.

It is preferred if the refractive index of the first optically transparent layer (a) of the sensor platform as a solid support is greater than 1.8.

It is further preferred if the first optically transparent layer (a) of the sensor platform as a solid support comprises TiO₂, ZnO, Nb₂O₅, Ta₂O₅, HfO₂, or ZrO₂, preferably TiO₂ or Ta₂O₅ or Nb₂O₅.

The first optically transparent layer (a) preferably has a thickness of 40 to 300 nm, most preferably of 100 to 200 nm.

Characteristic of a further embodiment of the bioanalytical detection method according to the invention is that an additional optically transparent layer (b′) with lower refractive index than layer (a) and with a thickness of 5 nm-10 000 nm, preferably of 10 run-1000 nm, is located between the optically transparent layers (a) and (b) and in contact with layer (a). This intermediate layer (b′) can for example serve to improve the adhesion of layer (a) on layer (b) or to reduce the effect of surface roughnesses of layer (b). However, layer (b′) can also serve to reduce the penetration of the evanescent field of light guided in layer (a) into layer (b), for example in order to reduce an unwanted luminescence excitation in layer (b).

Characteristic of a preferred embodiment of the bioanalytical detection method according to the invention is that the in-coupling of excitation light into the optically transparent layer (a) to the measurement areas (d) on the sensor platform as a solid support is performed using one or more optical in-coupling elements from the group formed by prism couplers, evanescent couplers comprising joined optical waveguides with overlapping evanescent fields, butt-couplers with focusing lenses, preferably cylindrical lenses, arranged in front of a front face (distal end) of the waveguiding layer, and grating couplers.

It is especially preferred in this case that the in-coupling of excitation light into the optically transparent layer (a) to the measurement areas (d) is performed using one or more grating structures (c) that are formed in the optically transparent layer (a).

Subject of the invention in a more general form is a bioanalytical detection method according to any of the embodiments described above, wherein one or more liquid samples comprising vesicles generated from living cells (from a bioanalytical reagent according to the invention) with associated receptors are brought into contact with the ligands for these receptors immobilized in one or more measurement areas, and a signal change caused by a binding of the receptors associated with said vesicles to their immobilized ligands is measured.

It is preferred in this case if the signal transduction of receptors associated with vesicles generated from living cells (from a bioanalytical reagent according to the invention), is measured after binding of these receptors to their immobilized ligands, wherein this signal transduction may be triggered, for example, by binding of further ligands to the receptors associated with said vesicles or by other inducing influences.

For example, in multiple measurement areas a uniform kind of ligand may be immobilized. If these measurement areas individually or several of these measurement together can be fluidically addressed, for example, within sample compartments comprising the solid carrier (support) as a base plate (see also below), screening methods based on the use of a bioanalytical reagent according to the invention are facilitated that are interesting for industrial research and development. For example, different vesicles generated from different living cells, with optionally different associated receptors and optionally different additional biological compound (components) may be supplied to different measurement areas with similar ligands immobilized therein, and the different binding behavior to the immobilized ligands in the discrete measurement areas may be investigated.

Characteristic of a further preferred embodiment of this bioanalytical detection method according to the invention is that the binding of receptors that are associated with vesicles generated from living cells (from a bioanalytical reagent according to the invention) to said immobilized ligands occurs in competition with the binding of these receptors associated with said vesicles to ligands in free solution.

If these measurement areas individually or several of these measurement areas together can be fluidically addressed, for example, within sample compartments comprising the solid carrier (support) as a base plate (see also below), it is also possible to supply to individual sample compartments different concentrations of vesicles generated from living cells in a bioanalytical reagent according to the invention, and to investigate the competition between the binding of the associated receptors to the immobilized ligands and to ligands located in free solution. Another variant comprises generating different surface concentrations of ligands immobilized in measurement areas, to which a uniform concentration of vesicles in a bioanalytical reagent according to the invention is added, and then to investigate again the competition between the binding of the associated receptors to the immobilized ligands and to the ligands in free solution.

It is obvious for those skilled in the art that a variety of further possible embodiments results from the combination of the aforementioned variants of bioanalytical detection methods according to the invention.

The aforementioned variants using ligands immobilized on a solid carrier (support) to which vesicles generated from living cell, from a bioanalytical reagent according to the invention, are supplied in a sample, are especially well-suited for determinations based on a change in the mass coverage of the solid carrier (support) (such as refractive methods based on a change in the effective refractive index on the surface of an optical waveguide, see below). For other applications it is advantageous to immobilize vesicles generated from living cells, from a bioanalytical reagent according to the invention, on a solid carrier (support) and then to address them (optionally in an individually addressable way for different measurement areas) with one or more samples comprising ligands to be detected. It is obvious to a person skilled in the art that embodiments analogous to those variants of embodiments described above result from this inversion of the assay architecture.

It is characteristic of a large group of embodiments of the bioanalytical detection method according to the invention that one or more liquid samples are brought into contact with the vesicles which are generated from living cells (from a bioanalytical reagent according to the invention) and immobilized in one or more measurement areas, along with their associated receptors, and that a signal change resulting from the binding of ligands to said receptors or from other inducing influences on said receptors is measured.

Characteristic of a preferred embodiment is that one or more liquid samples are brought into contact with the vesicles which are generated from living cells (from a bioanalytical reagent according to the invention) and immobilized in one or more measurement areas, along with their associated receptors, and that the signal transduction of those receptors resulting from the binding of ligands to said receptors or from other inducing influences on said receptors is measured.

For a special embodiment it is characteristic that the binding of ligands from a supplied sample to receptors that are associated with the immobilized vesicles generated from living cells (from a bioanalytical reagent according to any of claims 1-33) occurs in competition with the binding of these ligands to receptors in free solution which are optionally associated with vesicles.

Characteristic of a specially preferred embodiment is that one or more liquid samples, comprising vesicles generated from living cells (from a bioanalytical reagent according to the invention) with associated receptors, are brought into contact with the ligands for these receptors, the ligands being immobilized in one or more measurement areas, excitation light from one or more light sources of similar or different wavelengths is in-coupled to the measurement areas (d) by one or more grating structures (c), and the change of optical signals emanating from one or more measurement areas (d), caused by a binding of the receptors associated with said vesicles to their immobilized ligands, is measured.

Characteristic of another specially preferred embodiment is that one or more liquid samples are brought into contact with the vesicles immobilized in one or more measurement areas (d), along with their associated receptors, excitation light from one or more light sources of similar or different wavelengths is in-coupled to the measurement areas (d) by one or more grating structures (c), and the change in optical signals emanating from one or more measurement areas (d), caused by the binding of the ligands to said receptors or by other inducing influences on said receptors, is measured.

These variants lead to various further possible embodiments of the bioanalytical detection method according to the invention. Characteristic of one group of embodiments is that said changes in optical signals from the measurement areas (d) are caused by changes in the effective refractive index in the near-field of the optically transparent layer (a) in these measurement areas and are measured at the actual excitation wavelength.

Characteristic of another preferred group of possible embodiments of the bioanalytical detection method according to the invention is that said changes in optical signals from the measurement areas (d) are changes in one or more luminescences of similar or different wavelength which have been excited in said measurement areas in the near-field of the optically transparent layer (a), and which are measured each at a wavelength different from the corresponding excitation wavelength.

It is preferred if the one or more luminescences and/or measurements of light signals at the excitation wavelength are determined polarization-selectively, wherein preferably the one or more luminescences are measured at a polarization that is different from the polarization of the excitation light.

Subject of the invention in an again more general form is a bioanalytical detection method according to any of the aforementioned embodiments for the simultaneous or sequential, quantitative and/or qualitative determination of one or more analytes from the group of receptors or ligands, chelators or “histidine tag components”, enzymes, enzyme co-factors or inhibitors.

It is characteristic of the bioanalytical detection method according to any of the aforementioned embodiments that the samples to be examined are, for example, aqueous solutions or surface water or soil or plant extracts or bio- or process broths, or are taken from biological tissue fractions or from food, or odorous or flavoring substances or cosmetic compounds.

A further subject of the invention is a solid carrier (support) comprising, immobilized on a surface, at least one vesicle generated from a living cell, from a bioanalytical reagent according to the invention and any of the aforementioned embodiments, said vesicle comprising at least one receptor characterized by the fact that a mechanism of signal transduction triggered by said receptor in the said living cell used for vesicle generation is preserved in said vesicle after immobilization of the vesicle.

It is characteristic of a preferred embodiment of the solid carrier (support) according to the invention that vesicles, each comprising at least one receptor, are immobilized in discrete measurement areas (d) with one or more vesicles each.

In this case, it is preferred if vesicles with at least two different kinds of receptor are immobilized in multiple measurement areas (d), wherein vesicles with a uniform kind of receptor are preferably immobilized in each case within an individual measurement area.

The one or more vesicles generated from a living cell may be immobilized on the surface of said solid support for example by means of covalent binding or physical adsorption (electrostatic or van der Waals interaction or hydrophilic or hydrophobic interaction or a combination of these interactions).

It is preferred if an adhesion-promoting layer is deposited between the surface of said solid support and the one or more vesicles immobilized thereon. According to the invention, the adhesion-promoting layer is designed in such a way that a mechanism of signal transduction triggered by the one or more receptors in said living cell is preserved also after immobilization of the vesicles generated from a living cell, from a bioanalytical reagent comprising at least one receptor, on said adhesion-promoting layer.

The adhesion-promoting layer preferably has a thickness of less than 200 nm, most preferably of less than 20 nm.

The adhesion-promoting layer may comprise a chemical compound of the group of silanes, epoxides, functionalized, charged or polar polymers and “self-organized functionalized mono or multiple layers”.

Characteristic of a preferred embodiment is that the adhesion-promoting layer comprises a monomolecular layer of mainly one kind of protein, such as serum albumins or streptavidin, or of modified proteins, such as biotinylated serum albumin.

Characteristic of another preferred embodiment is that the adhesion-promoting layer comprises self-organized alkane-terminated monolayers of mainly one kind of chemical or biochemical molecule.

Especially preferred is an embodiment of which it is characteristic that the adhesion-promoting layer is provided as a double layer (bilayer) comprising an initial self-organized alkane-terminated anchoring layer and a second layer formed by self-organization (self-assembly) of synthetic or natural lipids.

The one or more vesicles generated from a living cell may be immobilized on the surface of said solid carrier (support), for example, by means of covalent binding or physical adsorption (electrostatic or van der Waals interaction or hydrophilic or hydrophobic interaction or a combination of these interactions).

A specific embodiment of the solid carrier (support) according to the invention, using a very specific variant of vesicle immobilization, comprises association with the adhesion-promoting layer of biological or biochemical or synthetic recognition elements which recognize and bind a vesicle generated from a living cell with surface-associated biological or biochemical or synthetic components for specific recognition and binding, as part of the corresponding specific embodiment of a bioanalytical reagent according to the invention described above. These specific interactions for the recognition and binding of the vesicles to their recognition elements on the adhesion-promoting layer may for example be based on interactions with biotin/streptavidin, so-called “histidine tags”, sugars or peptide affinity interactions, wherein any one of the two binding partners in each case may be associated with the vesicle surface and the other anchored on the surface of said adhesion-promoting layer.

Characteristic of another embodiment of the solid carrier (support) according to the invention is that at least one ligand for a receptor, which is bound to a vesicle generated from a living cell, from a bioanalytical reagent according to the invention, is immobilized, optionally by means of a spacer molecule, on the surface of the solid carrier (support).

It is preferred in this case if at least two different ligands for receptors which are bound to a vesicle generated from a living cell, from a bioanalytical reagent according to the invention, are immobilized in multiple measurement areas (d), wherein preferably a uniform kind of ligand is immobilized within an individual measurement area.

Said ligands may be immobilized on the surface of the solid carrier (support) by means of covalent binding or physical adsorption (e.g. electrostatic or van der Waals interaction or hydrophilic or hydrophobic interaction or a combination of these interactions).

It is preferred if an adhesion-promoting layer is applied between the surface of the solid carrier (support) and said ligands immobilized thereon.

For selection of the adhesion-promoting layer for immobilization of said ligands, the same preferences are applicable as those mentioned above for an adhesion-promoting layer for immobilization of vesicles generated from a living cell, from a bioanalytical reagent according to the invention.

Characteristic of a particularly preferred embodiment of a solid carrier (support) according to the invention is that regions between the laterally separated measurement areas, with vesicles generated from living cells (from a bioanalytical reagent according to any of the described embodiments) immobilized in these measurement areas, or with ligands for receptors that are bound to vesicles generated from living cells (from a bioanalytical reagent according to any of the described embodiments), and/or regions within these measurement areas, between the compounds immobilized therein, are “passivated” in order to minimize nonspecific binding of analytes or of their detection reagents, i.e. that compounds which are “chemically neutral” towards the analyte are deposited between the laterally separated measurement areas (d) and/or within these measurement areas (d) between said immobilized compounds, the “chemically neutral” compounds preferably being composed of the groups that are formed by albumins, casein, detergents, such as Tween 20, detergent/lipid mixtures (of synthetic and/or natural lipids), synthetic and natural lipids or also hydrophilic polymers, such as polyethylene glycols or dextrans.

It is also possible to passivate an activated surface (activated for immobilization of the biological, biochemical or synthetic recognition elements), this surface comprising e.g. poly-L-lysin or functionalized silanes (e.g. comprising aldehyde or epoxy groups), for example by the addition of reducing reagents such as sodium borate (in the case of aldehyde or epoxy groups).

The material of the surface of the solid support (carrier) with immobilized vesicles generated from living cells (from a bioanalytical reagent according to the invention and any of the described embodiments), or with immobilized ligands for receptors that are bound to vesicles generated from living cells (from a bioanalytical reagent according to the invention and any of the described embodiments), may comprise a material of the group which is formed e.g. by moldable, sprayable or millable plastics, carbon compounds, metals, such as gold, silver, copper, metal oxides or silicates, such as glass, quartz or ceramics, or silicon or germanium or ZnSe or a mixture of these materials.

In this case, said solid carrier (support) may be provided in a variety of different embodiments. It may be provided e.g. as a glass or microscope plate. It may also be a microtiter plate of the type that is, for example in widespread use for screening assays (for testing numerous compounds, e.g. using classical fluorescence methods or fluorescence correlation spectroscopy).

It is preferred if the surface of said solid support (carrier) is essentially planar.

Characteristic of a preferred group of embodiments of the solid carrier (support) according to the invention is being provided as an optical or electronic sensor platform.

Another subject of the invention is therefore a sensor platform as a solid support (carrier) according to any of the aforementioned embodiments, wherein said solid support is transparent at least in a region of wavelengths in the ultraviolet to infrared spectrum and preferably comprises a material from the group that is formed e.g. by moldable, sprayable or millable plastics, carbon compounds, metals, metal oxides or silicates, such as glass, quartz or ceramics, or silicon or germanium or ZnSe or a mixture of these materials.

It is preferred in this case if said sensor platform is a solid carrier (support) wherein an optical waveguide serves as sensor platform.

Characteristic of a preferred embodiment of the sensor platform used as a solid carrier (support) is an optical thin-film waveguide serving as a sensor platform, with an initial optically transparent layer (a) with refractive index n₁ on a second optically transparent layer (b) with refractive index n₂, wherein n₁>n₂.

For many applications such an embodiment of a sensor platform according to the invention used as solid carrier (support) is advantageous when it is divided into two or more discrete waveguiding regions.

The material of the second optically transparent layer (b) of the sensor platform as a solid support may be selected from the group that is formed by silicates, such as glass or quartz, or transparent moldable, sprayable or millable, especially thermoplastic plastics, such as polycarbonates, polyimides, polymethyl methacrylates, or polystyrenes.

It is preferred if the refractive index of the first optically transparent layer (a) of the sensor platform as a solid support is greater than 1.8.

For numerous applications such an embodiment of a sensor platform according to the invention, used as a solid carrier (support), is preferred when the first optically transparent layer (a) comprises a material of the group of TiO₂, ZnO, Nb₂O₅, Ta₂O₅, HfO₂, or ZrO₂, preferably of TiO₂ or Ta₂O₅ or Nb₂O₅.

The first optically transparent layer (a) preferably has a thickness of 40 to 300 nm, most preferably of 100 to 200 nm.

Characteristic of a further embodiment of the sensor platform according to the invention, used as solid carrier (support) is that an additional optically transparent layer (b′) with lower refractive index than layer (a) and with a thickness of 5 nm-10 000 nm, preferably of 10 nm-1000 nm, is located between the optically transparent layers (a) and (b) and in contact with layer (a).

It is preferred if the in-coupling of excitation light into the optically transparent layer (a) to the measurement areas (d) is performed using one or more optical in-coupling elements from the group formed by prism couplers, evanescent couplers comprising joined optical waveguides with overlapping evanescent fields, butt-couplers with focusing lenses, preferably cylindrical lenses, arranged in front of a front face (distal end) of the waveguiding layer, and grating couplers.

In this case, it is especially preferred if the in-coupling of excitation light into the optically transparent layer (a) to the measurement areas (d) is performed using one or more grating structures (c) that are formed in the optically transparent layer (a).

Characteristic of an improvement of a sensor platform according to the invention and any of the aforementioned embodiments is that they additionally comprise one or more sample compartments with said sensor platform as the base plate, said sample compartments being open towards the sensor platform at least in the region of the one or more measurement areas, wherein said sample compartments may be open or closed except for inlet and/or outlet openings at the side facing away from the sensor platform.

A variety of further embodiments of sensor platforms, which are suitable in combination with a bioanalytical reagent according to the invention and may be applied in a bioanalytical detection method according to the invention, are described in detail, for example, in patents U.S. Pat. No. 5,822,472, U.S. Pat. No. 5,959,292, and U.S. Pat. No. 6,078,705, and in patent applications WO 96/35940, WO 97/37211, WO 98/08077, WO 99/58963, PCT/EP 00/04869, and PCT/EP 00/07529. The embodiments of sensor platforms and methods for the detection of one or more analytes, as well as the optical and analytical systems described therein, are also a subject of the present invention, as part of sensor platforms according to the invention, as solid carriers. (supports) comprising a bioanalytical reagent according to the invention, and as parts of bioanalytical detection methods according to the invention which are performed therewith.

A further subject of the present invention is the use of a vesicle as a component of a bioanalytical reagent according to the invention and any of the aforementioned embodiments and/or of a solid carrier (support) according to the invention, comprising one or more vesicles immobilized thereon, as described in any of the aforementioned embodiments for the enrichment of membrane receptors or for the enrichment of proteins (such as antigens) triggering an immunological response in a two- or three-dimensional phase, which may then e.g. be administered to living organisms (e.g. to stimulate immune defense processes).

A further subject of the invention is the use of a vesicle as a component of a bioanalytical reagent according to the invention, as described in any of the aforementioned embodiments, as a compartment for therapeutic, diagnostic, photosensitive or other biologically active compounds for administration to a living organism.

The present invention also comprises the use of a bioanalytical reagent according to the invention as described in any of the aforementioned embodiments and/or of a solid carrier (support) according to the invention as described in any of the aforementioned embodiments, comprising one or more immobilized vesicles, and/or of a bioanalytical detection method according to the invention as described in any of the aforementioned embodiments for investigating receptor-ligand interactions, especially for determining the binding strength and kinetic parameters of these interactions between a receptor and its ligand, or for determining the channel activity of an ion channel receptor after ligand binding or other inducing influences on said receptor, or for determining the enzymatic activity of enzymes associated with a vesicle, as a component of a bioanalytical reagent according to the invention, or for determining secondary messenger compounds after ligand binding to a receptor resulting in a signal transduction, or for determining protein-protein interactions, or for determining protein kinases.

The present invention additionally comprises the use of a bioanalytical reagent according to the invention as described in any of the aforementioned embodiments and/or of a solid carrier (support) according to the invention as described in any of the aforementioned embodiments, comprising one or more immobilized vesicles, and/or of a bioanalytical detection method according to the invention as described in any of the aforementioned embodiments for quantitative and/or qualitative analyses for determining chemical, biochemical or biological analytes in screening methods in pharmaceutical research, combinatorial chemistry, clinical and pre-clinical development, for real-time binding studies and for determining kinetic parameters in affinity screening and in research, for qualitative and quantitative analyte determinations, especially for DNA and RNA analytics, for generation of toxicity studies and for the determination of expression profiles, and for determining antibodies, antigens, pathogens or bacteria in pharmaceutical product development and research, human and veterinary diagnostics, agrochemical product development and research, for symptomatic and pre-symptomatic plant diagnostics, for patient stratification in pharmaceutical product development and for therapeutic drug selection, for determining pathogens, nocuous agents and germs, especially of salmonella, prions and bacteria, in food and environmental analytics, and for analysis and quality control of odorous and flavoring substances.

### EXAMPLES

The present examples describe the preparation of a bioanalytical reagent according to the invention with at least one vesicle which has been generated from a living cell, comprising at least one receptor, wherein a mechanism of signal transduction triggered by this receptor in the living cell used remains preserved in said vesicle as part of the bioanalytical reagent.

In these examples, it is shown
that membrane receptors of membranes of living cells can be transferred to membranes of “native” vesicles without loss of function
that components from the lumen of the vesicle-forming cell can be transferred to the “native” vesicles formed without an exchange with the surrounding medium
that luminescence indicators required for secondary responses can be incorporated into “native” vesicles
and that the transferred components including the indicators remain stable within the interior of “native” vesicles.

### Example 1

### Preparation of “Native” Vesicles as Component of a Bioanalytical Reagent According to the Invention and Visualization of the Prepared Vesicles

1.1. Preparation of Vesicles of Defined Size

For the preparation of vesicles (“vesiculation process”) adherent growing HEK293 (human embryonic kidney) cells were cultured in each case in 15 ml DMEM/F12 (Dulbecco's modified Eagle medium, Gibco BRL Life Technologies) in 75 ml T flasks (TPP, Switzerland). To the medium, 2.2% fetal calf serum (Gibco BRL Life Technologies) was added. The cell cultures were stored in an incubator (37° C., 5% CO₂).

To visualize the cytoplasmic contents of the cell during the vesiculation process, HEK293 cells were transfected with plasmid DNAs (Clontech; Palo Alto, Calif., USA) coding for Aequorea victoria GFP (Green Fluorescent Protein) using the customary method of calcium phosphate-DNA coprecipitation (Jordan M., Schallhorn A., Wurm F. M-: “Transfecting mammalian cells: optimization of critical parameters affecting calcium-phosphate precipitate formation”, Nucleic Acids Res. 24 (1996) 596-601). Twenty hours after transfection, the green fluorescence of GFP was visible in the entire cell contents, after excitation at 488 nm, using a confocal fluorescence microscope. This control procedure was used in particular to demonstrate that, during the vesiculation process, the cell and vesicle content remains intact, i.e. that there is no contact and no intermixing with the surrounding outer cell medium (see FIG. 1).

For vesiculation, the serum contained in the culture medium was removed by decantation or, in the case of the suspension cells, by centrifugation (rotor SS34 Sorvall, at 120 g; centrifugation time 5 minutes) and exchanged for serum-free DEMEM medium. The cell concentration lay between 1×10⁷ and 5×10⁷ cells per milliliter.

Cytochalasin B or D (Sigma-Aldrich) (stock solution with a concentration of 2 mg/ml in DMSO) was added to the preheated DMEM medium in a final concentration of 20 μg/ml. The vesiculation process was dependent on the cell age and cell line and lasted between 5 and 60 minutes. Shear forces were then applied (1 minute vortex) to separate off any vesicles remaining on the cell surface as completely as possible. This suspension was then passed through a sterile syringe filter (5 μm pore size; Acrodisc) to separate off any remaining cell bodies from the vesicles. The vesicle suspension was then concentrated by centrifugation (rotor SS 34, Sorvall, at 5400 r.p.m. (revolutions per minute; 20 minutes).

The resulting vesicle pellet was then resuspended in 500 μl phosphate buffer (pH 7.5) and loaded onto a preformed sucrose gradient. This gradient comprised three sucrose layers with decreasing density (in ascending order 2 M, 1.5 M, 1.3 M sucrose in deionized, sterile water). After 90 minutes of centrifugation at 25000 r.p.m. in a centrifuge rotor (TST 60.4 at 84,840 g; Sorvall; Kontron ultracentrifuge) three clearly separated bands were visible. The bands contained vesicles of various sizes. The vesicle membranes were visualized by staining with octadecyl rhodamine B chloride (R18) (Molecular Probes Inc., USA) in order to measure the size of the vesicles in a confocal fluorescence microscope (excitation: 560 nm/emission: 590 nm) using this fluorescence labeling. The uppermost band contained vesicles with a diameter of 100-300 nm, the middle band contained vesicles with a diameter of 500-800 nm and the lowest band contained vesicles with a size distribution between 1 μm and 3 μm.

The “native” vesicles produced in this way were thus smaller than, for example, red blood cells (3-5 μm). The vesicles from the two upper bands were even smaller than mitochondria.

As shown in the following, the “native vesicles” contain parts of the endoplasmic reticulum. In view of their size, however, the cell nucleus and mitochondria (in vesicles with a diameter of less than 800 nm) are excluded from incorporation into the vesicles. In the case of G-protein-coupled receptors (GPCR) it follows from this size distribution, for example, that the vesicles can only contain endoplasmic reticulum, albeit as the most important reservoir of intracellular calcium ions and as essential component of the GPCR-signal transduction cascade.

Vesicles from the uppermost band were used for experiments aimed at investigating the residual capacity of receptors for ligand binding following preparation, whereas vesicles from the two lower fractions were used for detecting the release of secondary messenger compounds, such as Ca²⁺ and cAMP, because these vesicles can be charged to a greater extent with suitable fluorescence indicators.

The process of budding and pinching of cells in vesicle formation is schematically illustrated in the diagram below the fluorescence microscopy images (FIG. 1): (a) Normal actin filament network in cell cortex. (b) The actin filaments retract at certain points for some minutes after administration of cytochalasin B/D (concentration 20 μg/ml), and the endoplasm of the cell can expand locally, resulting in (c) cell budding and pinching.

1.2. Determination of Endoplasmic Reticulum in “Native” Vesicles

For determination of the endoplasmic reticulum in native vesicles, as a leading precondition for the release of secondary Ca²⁺ after activation of the signal transduction cascade of G-protein-coupled receptors, the green fluorescent protein (GFP) of Aequorea Victoria at the level of the coding DNA was furnished with a peptide signal sequence (MRLCIPQVLLALFLSMLTAPGEG) which, during the synthesis of GFP in the cell, guides it into the endoplasmic reticulum. This molecular biological intervention did not have any negative influence on the vitality of the cultivated HEK293 cells. The overlapping of GFP fluorescence with the geometric dimensions of the endoplasmic reticulum (ER) was studied using commercial lipophilic tracer for ER (1,1′-dihexadecyl-3,3,3′,3′-tetramethylindocarbocyanine perchlorate (DiIC16(3); Molecular Probes) in whole cells. Based on the fluorescence of the GFP molecules, parts of the endoplasmic reticulum were demonstrated in native vesicles using confocal microscopy images (FIG. 2).

1.3. Preparation and Determination of “Native” Vesicles with (A) Incorporated, Fluorescence-Labeled G-Protein and (B) Incorporated Indicators

(A) “Native” vesicles were prepared with the recombinant G-alpha subunit of a G-protein (G_{α}). To demonstrate the presence of this protein in living HEK293 cells using a confocal fluorescence microscope, the G-alpha-15 protein (G_{α15}) at the level of the coding DNA was fused with Aequorea Victoria GFP. For this purpose the DNA coding for EGFP (Enhanced Green Fluorescent Protein; Clontech) was inserted in the G_{αq} subunit. The resulting fusion protein was transfected into HEK293 cells using the above-mentioned calcium phosphate precipitation method. 24 hours after transfection, the green-labeled G_{α}-protein was detectable in the cytoplasm (as in FIG. 1a of Example 1.1). The recombinantly expressed proteins were localized close to the cell membrane using confocal microscopy.

After cytochalasin B (2 mM) was added to the HEK293 cells expressing G protein, plasma membrane vesicles which had incorporated green fluorescent G_{α} fusion protein were pinched off.

(B) “Native” vesicles were prepared with incorporated, chemical fluorescence indicators. For this purpose, HEK cells were loaded with calcium-sensitive indicators (Fura Red, C₄₇H₅₂N₄O₂₄S, 149732-62-7 glycine, N-[2-[(acetyloxy)methoxy]-2-oxoethyl]-N-[5-[2-[2-[bis[2-[(acetyloxy)methoxy]-2-oxoethyl]amino]-5-methylphenoxy]ethoxy]-2-[(5-oxo-2-thioxo-4-imidazolidinylidene)methyl]-6-benzofuranyl]-, (acetyloxy)methyl ester and Fluo-3/AM (C₃₆H₄₅Cl₂N₇O₁₃; 121714-22-5 glycine, N-[4-[6-[(acetyloxy)methoxy]-2,7-dichloro-3-oxo-3H-xanthen-9-yl]-2-[2-[2-[bis[2-[(acetyloxy)methoxy]-2-oxyethyl]amino]-5-methylphenoxy]ethoxy]phenyl]-N-[2-[(acetyloxy)methoxy]-2-oxyethyl]-, (acetyloxy)methyl ester; Molecular Probes). Per milliliter of cell nutrient culture medium (DMEM/F12: Dulbecco's modified Eagle Medium F12) 12.5 μg Fluo-3/AM and 24.5 μg Fura Red were added. The ion-sensitive indicators were dissolved beforehand in DMSO. Loading with acetoxymethyl ester lasted 60 minutes at 37° C. Addition of cytochalasin led to pinching-off of vesicles with a marked indicator load. Both indicators were clearly visible under fluorescence microscopy. The concentration of free Ca 2+ions in the interior of the vesicles and also the concentration of free Ca²⁺ ions in the cytosol of the cell from which they originated were determined by analyzing the measured fluorescence intensities. The two were comparable and amounted to 130 nM.

1.4. Preparation of Vesicles with Incorporated, Recombinant Fusion Proteins from G Proteins and Fluorescence-Labeled Proteins as well as Incorporated Indicators

The DNA coding for EGFP (Enhanced Green Fluorescent Protein; Clontech) was inserted in the G_{αq} subunit. The resulting fusion protein was transfected into HEK293 cells using the above-mentioned calcium phosphate precipitation method. 24 hours after transfection, the green labeled G_{α} protein was detectable in the cytoplasm and showed, in addition to the occurrence of diffuse areas, a frequent tendency towards the formation of aggregates (0.5-1 μm in diameter) close to the plasma membrane. After cytochalasin B (2 mM) was added to the HEK293 cells expressing G protein, plasma membrane vesicles which had incorporated the green fluorescent G_{α} fusion protein were pinched off (FIG. 3). The cells were loaded with calcium-sensitive indicators (Fura Red and Fluo-3/AM). For every milliliter of cell nutrient culture medium (DMEM/F12: Dulbecco's modified Eagle Medium F12) 12.5 μg Fluo-3/AM and 24.5 μg Fura Red were added. The ion-sensitive indicators were dissolved beforehand in DMSO. Loading with acetoxymethyl ester lasted 60 minutes at 37° C. Addition of cytochalasin led to pinching-off of indicator-loaded vesicles. Both indicators were clearly visible in the vesicle lumen on fluorescence microscopy (FIG. 3). Analysis of the measured fluorescence intensities showed a concentration of free Ca²⁺ ions amounting to 130 nM, as is also measured in the determination of free Ca²⁺ ions in the cytosol of a cell.

### Example 2

### Preparation of “Native” Vesicles Capable of Signal Transduction with Incorporated Receptors and the Detection Thereof

2.1. Incorporation of Ion Channel Receptors in “Native” Vesicles with the Preservation of Their Functionality, as Illustrated in the 5HT_{3A} Receptor

Inn the following examples, the 5HT₃ serotonin receptor is used as a representative ligand-controlled ion channel. In the literature, two different types of 5HT₃ receptor are described, the 5HT_{3A} and the 5HT_{3B} receptor (Davies P. A., Pistis M., Hanna M. C., Peters J. A., Lambert J. J., Hales T. G., Kirkness E. F., “The 5-HT_{3B} subunit is a major determinant of serotonin-receptor function”, Nature 397 (1999) 359-363). In the following examples, only the 5HT_{3A} receptor is used.

Various recombinant constructs of the serotonin (5HT_{3A}) receptor were expressed in HEK293 cells under human cytomegalovirus gene promoter control. Transient expression of the full wild-type receptor was achieved by co-transfection of the eukaryotic expression vector CMVβ (Clontech, Palo Alto, Calif.) with the receptor of coding cDNA and/or with corresponding cDNA for cytosolic GFP, in order to identify cells which expressed the serotonin receptor (5-HT_{3A} R) at the same time.

16 to 20 hours before transfection, HEK293 cells (10⁵ cells/ml) were seeded in 6-well plates or, in the case of samples for later investigation under confocal fluorescence microscopy, on sterile cover glasses (diameter 22 mm) in 6-well plates. The cells were transfected using Effectene (Qiagen, Hilden, Germany) in accordance with the manufacturer's instructions. After four hours' transfection in a humid atmosphere (5% CO₂, 37° C.) the transfection medium was exchanged with fresh cell culture medium.

Vesicles were prepared from the cells 48 hours after transfection in a manner similar to that described in Example 1.1. For this purpose, the cells were exposed to a trypsin-EDTA solution (Sigma) in two 6-well plates (0.5-1 ml per well) for one to two minutes with gentle stirring. The well contents were then centrifuged for 5 minutes (1200 r.p.m., Rotor SLA-600, Sorvall, Newtown, USA). The supernatant was discarded and the resulting pellet resuspended in 10 ml in PBS buffer (10 mM phosphate buffer solution with Na₂HPO₄, K₂HPO₄, 138 mM NaCL, 2.7 mM KCl, pH 7.4) in a vortex mixer. The generation of vesicles from the cell preparation was triggered by the addition of cytochalasin from a stock solution in DMSO (4 mg/ml) up to a final concentration of 20 μg/ml. The resulting pinched-off vesicles were separated from the cells by shear forces applied again by means of a vortex (2 minutes), before the suspension was finally resuspended and the supernatant then passed through a syringe filter (1.2 μm pore size, Acrodisc) with the vesicles contained therein.

According to the above description, “native” vesicles were prepared with expressed receptor contained therein. The presence of the 5HT_{3A} receptor in the outer vesicle membrane was detected by labeling of this receptor with the receptor-specific ligand GR-Cy5 (=1,2,3,9-tetrahydro-3-[(5-methyl-1 h-imidazol-4-yl)methyl]-9-(3-amino-(N-Cy5-amide)-propyl)-4H-carbazol-4-one). Intensity profiles of the fluorescence from fluorescence-labeled receptors and of GFP, taken up by the vesicles from the GFP-labeled cytosolic cell contents, unequivocally demonstrate the existence of the receptor in the “native” vesicle membrane and thus its origin from the plasma membrane of the vesicle-forming mammalian cell. The presence of GFP in the “native” vesicle unequivocally demonstrates the cytosolic origin from the cell (FIG. 4).

2.1.1 Determination of Ligand-Binding Capacity of the 5HT_{3A} Receptor in “Native” Vesicles in Solution

The capacity of 5HT_{3A} receptors for specific ligand binding, as their “primary” functionality, after incorporation of the receptors in “native” vesicles was investigated by means of a competitive radiological binding assay (Tairi A. P., Hovius R., Pick H., Blasey H., Bernard A., Surprenant A., Lundstrom K., Vogel H., “Ligand binding to the serotonin 5HT₃ receptor studied with a novel fluorescent ligand”, Biochemistry 37 (1998) 15850-15864; Wohland T., Friedrich K., Hovius R., Vogel H., “Study of ligand-receptor interactions by fluorescence correlation spectroscopy with different fluorophores: evidence that the homopentameric 5-hydroxytryptamine type 3As receptor binds only one ligand”, Biochemistry 38 (1999) 8671-8681).

To this end, samples of said reagent were incubated with the vesicles in solution for 60 minutes at room temperature with 1.5 nM of the tritium-labeled ligand [³H]-GR65630 in 240 μl HEPES buffer (10 mM HEPES, pH 7.4) (HEPES: 4-(2-hydroxyethyl)piperazine-1-ethanesulfonic acid) in 96-well MultiScreen plates (Millipore, F-Molsheim). The incubation was completed by rapid filtration followed by washing 3 times with 300 μl of ice-cold HEPES buffer each time. The filters were transferred to scintillation vessels and each taken up in 1 ml of Ultima Gold TM (Packard, meridan, USA). The radioactivity was measured using a TriCarb 2200CA liquid scintillation counter. The extent of nonspecific binding to the receptor and to the vesicles was estimated in the presence of a high surplus of quipazine (1 μM). The dissociation constant K_{d} was determined from Scatchard plots in 6 different concentrations of the ligand [³H]-GR65630. The binding behavior (“pharmacology”) of the receptor was determined in the form of IC₅₀ values (concentration at 50% inhibition) through the competition of various pharmacologically active substances with the binding of the radioactively labeled reference ligand [³H]-GR65630. All experiments were carried out in duplicate. For comparison purposes, experiments were also conducted under comparable conditions in the whole mother cells. The following table summarizes the experimentally determined dissociation constants.

| | Receptor in | Receptor |
|---|---|---|
| | mother cell | in “native” vesicles |
| | pKᵢ | pKᵢ |
| Antagonist | | |
| GR-H | 10.2 ± 0.1 | 9.7 ± 0.1 |
| Granisetron | 9.2 ± 0.1 | 9.5 ± 0.1 |
| Ondansetron | 8.5 ± 0.1 | 8.6 ± 0.1 |
| Agonist | | |
| Quipazine | 9.3 ± 0.1 | 9.4 ± 0.2 |
| mCPBG | 8.2 ± 0.1 | 8.3 ± 0.1 |
| 5HT | 7.5 ± 0.1 | 7.7 ± 0.1 |
| PBG | 6.5 ± 0.1 | 6.6 ± 0.1 |
| [³H]-GR65630 | 9.0 ± 0.1 | 9.4 ± 0.1 |

Explanation of abbreviated names:

| Antagonist | |
|---|---|
| GR-H | 1,2,3,9-tetrahydro-3-[(5-methyl-1H-imidazol- |
| | 4-yl)methyl)]-9-(3-aminopropyl)-4H-carbazol-4-one |
| Granisetron: | endo-N-(9-methyl-9-azabicyclo[3.3.1]non-3-yl)- |
| | 1-methyl-1H-indazole-3-carboxamide hydrochloride |
| Ondansetron: | 1,2,3,9-tetrahydro-9-methyl-3-[(2-methyl-1H-imidazol-1- |
| | yl)methyl]-4H-carbazol-4-one |
| Agonist: | |
| Quipazine: | 2-(1-Piperazinyl)quinoline |
| m-CPBG: | m-Chlorophenylbiguanide |
| 5-HT: | 5-Hydroxytryptamine |
| PBG: | Phenylbiguanide |
| [³H]-GR65630 | see: Kilpatrick G. J., Jones G. J., Tyers M. B., (1988) |
| | “The distribution of specific binding of the 5-HT3 |
| | receptor ligand [³H]-GR65630 in rat brain using |
| | quantitative autoradiography”, Neuroscience Letters |
| | 94 (1988) 156-160. |

2.1.1 Determination of Ligand-Binding Capacity of the 5HT_{3A} Receptor in Surface-Immobilized “Native” Vesicles

The ligand-binding experiments in surface-immobilized vesicles were carried out using the fluorescence-labeled ligand GR-Cy5. For these experiments, a homologous serotonin receptor construct 5HT_{3A} was used that was expressed with an EGFP (Enhanced Green Fluorescent Protein, Clontech) fused at the end of a subunit. The prepared “native” vesicles were immobilized on cover glasses by means of physical adsorption of the vesicles to the glasses, by two-hour or overnight incubation of typically 400 μl of a vesicle preparation solution prepared according to the above method in 6-well plates with cover glasses on the bottom at 4° C. The cover glasses were then removed from the plates with the vesicles immobilized thereon and inserted into an open cell, which was then filled with 200-300 μl PBS buffer.

The affinity of a fluorescent ligand for the vesicle-bound 5HT_{3A} receptors, in an experiment with a large number of vesicles, was studied by incubating a series of cover glasses for 2 hours each with vesicles in solution and increasing concentrations of fluorescent ligands at room temperature. A second series of cover glasses was incubated under the same conditions, for estimating the extent of nonspecific binding of the fluorescent ligands to the vesicles, likewise with vesicles in solution and the same increasing concentrations of fluorescent ligands, as well as 5 μM quipazine, as competitor in a high (>150-fold) surplus concentration.

The affinity of the fluorescent ligand for receptors bound to individual, discrete vesicles was determined by sequential addition of ascending concentrations of the fluorescent ligand to one and the same cover glass with vesicles immobilized thereon by overnight incubation. The studies were carried out in a manner analogous to that described above for a large number of vesicles.

The fluorescence intensities of the ligand were measured with a confocal fluorescence microscope (Zeiss, Laser Scanning Microscope LSM510), using a suitable filter set. The fluorescence signal of the GFP additionally incorporated into the vesicles was used in each case to adjust the microscope to the working distance of the plane in which the vesicles lay before the ligand was added. The fluorescence signals of the ligands (red) were referenced via the fluorescence signals of the EGFP (green) according to the number of active receptors per vesicle. The images presented were obtained from the signals of the fluorescence microscope recorded with a photomultiplier. For the image analysis, so-called “Regions of Interest” (ROIs) of the images were defined, the dimensions of which were adjusted to the areas to be measured.

The results of this experiment show that vesicles show differing numbers of associated 5HT_{3A} receptors, with the consequence of markedly differing fluorescence intensities of different vesicles, but the binding constants showed identical values in agreement with the values which have been reported in the literature and which were determined with isolated receptors or in complete cells (Tairi A. P., Hovius R., Pick H., Blasey H., Bernard A., Surprenant A., Lundstrom K., Vogel H., “Ligand binding to the serotonin 5HT₃ receptor studied with a novel fluorescent ligand”, Biochemistry 37 (1998) 15850-15864; Wohland T., Friedrich K., Hovius R., Vogel H., “Study of ligand-receptor interactions by fluorescence correlation spectroscopy with different fluorophores: evidence that the homopentameric 5-hydroxytryptamine type 3As receptor binds only one ligand”, Biochemistry 38 (1999) 8671-8681).

As an example, FIG. 5 shows the measured fluorescence intensities as a function of increasing concentrations of GR-Rho (1,2,3,9-tetrahydro-3-[(5-methyl-1H-imidazol-4-yl)methyl]-9-(3-amino-(N-rhodamineB-thiocarbamoyl)-propyl)-4H-carbazol-4-one) as fluorescent ligand (after subtraction of the fluorescence intensity F₀ observed in the absence of GR-Rho). The fit of measurement data with the Langmuir isotherm equation gave a value of K_{d}=(4.2+/−1.7) nM for the dissociation constant of the system comprising the HT₃ receptor and the GR-Rho ligand (FIG. 5).

2.1.3. Functional Capacity of the Full Signal Transduction Cascade of a Receptor Incorporated in “Native” Vesicles: Induction of Ion-Channel Activity (Secondary Function) of the 5HT_{3A} Receptor after Specific Ligand Binding (Primary Function).

It was surprisingly found that, with a bioanalytical reagent according to the invention, the full signal transduction transmitted via a receptor is preserved.

FIG. 6 demonstrates the ability of vesicles with incorporated 5HT_{3A} serotonin receptor to regulate the concentration of intracellular calcium. In this case, the original HEK cells from which the vesicles were obtained following the addition of cytochalasin according to the method described hereinabove were loaded with the acetoxymethyl ester of Fluo-3 as calcium indicator (generally 20 μg Fluo-3 in 500 μl DMEM/F12 medium). The prepared vesicles contained said fluorescent calcium indicator and parts of the endoplasmic reticulum in addition to the 5HT_{3A} receptor.

Fluorescence excitation of the Fluo-3 was performed at 488 nm. The emission was measured at 500-530 nm. The vesicles loaded with Fluo-3 were then stimulated with 350 μM m-chlorophenylbiguanide hydrochloride (mCPG), a receptor-specific agonist. Stimulation was performed by addition of the agonist to the vesicle medium (t=0 sec). The maximum fluorescence signal that occurred through binding of the calcium to the calcium indicator Fluo-3 was already measured less than one minute after addition of the agonist. The value amounted to 35 μM free calcium ions in the vesicle interior, i.e. in the cytosol surrounded by the vesicle. With the aid of calcium indicator Fluo-3, free calcium ions are detected only in the cytosol (vesicle lumen) transferred from the cell of origin. Calcium ions in the endoplasmic reticulum or in other cell organelles or parts of cell organelles possibly included in the vesicle are not determined by this detection method.

The increase in the fluorescence signal up to its maximum value after less than one minute and the subsequent decrease in the fluorescence signal to its baseline value after a further 2 minutes, i.e. after a total of 3 minutes following addition of the agonist, is a sign of the intact functional capacity of the complete mechanism of the signal transduction cascade within the vesicle, i.e. of the ability of the vesicle to “buffer” the concentration of free calcium ions. This includes the action of sodium-calcium exchangers, in addition to the timespan until closure of the receptor's ion channel and desensitization mechanisms characteristic of serotonin.

2.2. Incorporation of G-Protein-Coupled Receptors in “Native” Vesicles with the Preservation of Their Functionality, as Illustrated in the NK1 Receptor

An important member of the membrane-bound G-protein-coupled receptors (GPCRs) is the NK1 receptor. It plays an important immunological role in the activation of astrocytes in the central nervous system by substance P, a tachykinin. Tachykinins are neuropeptides which are active both in the peripheral and in the central nervous system and play a role in inflammatory processes, nociception and a number of autonomic reflexes.

There is evidence to suggest that GPCRs induce membrane ruffling (Okamoto H., Takuwa N., Yokomizo T., Sugimoto N., Sakurada S., Shigematsu H., Takuwa Y., “Inhibitory Regulation of Rac Activation, Membrane Ruffling, and Cell Migration by the G Protein-Coupled Sphingosine-1-Phosphate Receptor EDG5 but Not EDG1 or EDG3”, Mol Cell Biol 20 (2000) 9247-9261). This different behavior compared with that of the 5HT3 receptor described could influence incorporation in the native vesicles as a deleterious reagent. The following shows that this is not the case.

The NK1 receptor was fused with the DNA coding for EGFP (Clontech Palo Alto, Calif., USA) at the level of the coding DNA on the carboxy-terminal end and transfected into HEK293 cells by means of the calcium-phosphate/DNA co-precipitation method (Jordan M., Schallhorn A., Wurm F. M., “Transfecting mammalian cells: optimization of critical parameters affecting calcium-phosphate precipitate formation”; Nucleic Acids Res 24 (1996) 596-601). 24 hours after transfection, green labeled NK1 receptors were detectable in the plasma membrane of the HEK293 cells. Cytochalasin B (10 μg/ml) was then added to the expressing cells and vesicle formation observed under confocal microscopy. FIG. 7 demonstrates the incorporation of NK1-GFP fusion proteins in “native” vesicles. Using the NK1 receptor as a example, evidence is thus shown that G-protein-coupled receptors are incorporated in native vesicles.

3. Storage Life of Native Vesicles

Freshly prepared “native” vesicles could be stored for up to one week in phosphate buffer (PBS: 150 mM sodium phosphate, 150 mM NaCl, pH 7.2+0.1 (25° C.) or DMEM/F12 medium) under sterile conditions at 4° C. without any change in quality.

For freezing, “native” vesicles were resuspended in DMEM medium with 2.2% serum (fetal bovine serum) and 10% dimethylsulfoxide (DMSO). This vesicle suspension was shock-frozen in liquid nitrogen. Frozen vesicles could be stored up to 6 months at −80° C. without loss of quality. Compared with a freshly prepared vesicle fraction, frozen “native” vesicles did not show any significant changes in size or morphology after they were thawed out. They also did not show any increased tendency towards aggregation formation.

Determinations on the Stability of Frozen Vesicles:

Vesicles were prepared from HEK293 cells which expressed the GFP of Aquorea victoria in their cytoplasm. A fluorescent protein freely dissolved in cytoplasm should serve as evidence to show that the vesicles survive the freezing and thawing cycle intact and do not burst or drain. FIG. 8 shows an example of microscopy images of vesicles after 30 days' storage at −80° C. No damage or change to the morphology of the vesicles was observed.

### DESCRIPTION OF FIGURES

FIG. 1: Process of vesicles being pinched off with the transfer of cytoplasm from the cell into the vesicles: Images produced with a confocal fluorescence microscope showing HEK293 cells which, after transfection with Aequorea GFP, display a green fluorescence in the cytoplasm (excitation 488 nm/emission 510 nm); below, graphic illustrations of the process. (a) Normal actin-filament network in the cell cortex before the addition of cytochalasin. (b) After application of cytochalasin B, the cells begin to round off and form (c) bullous or pedunculate buds.

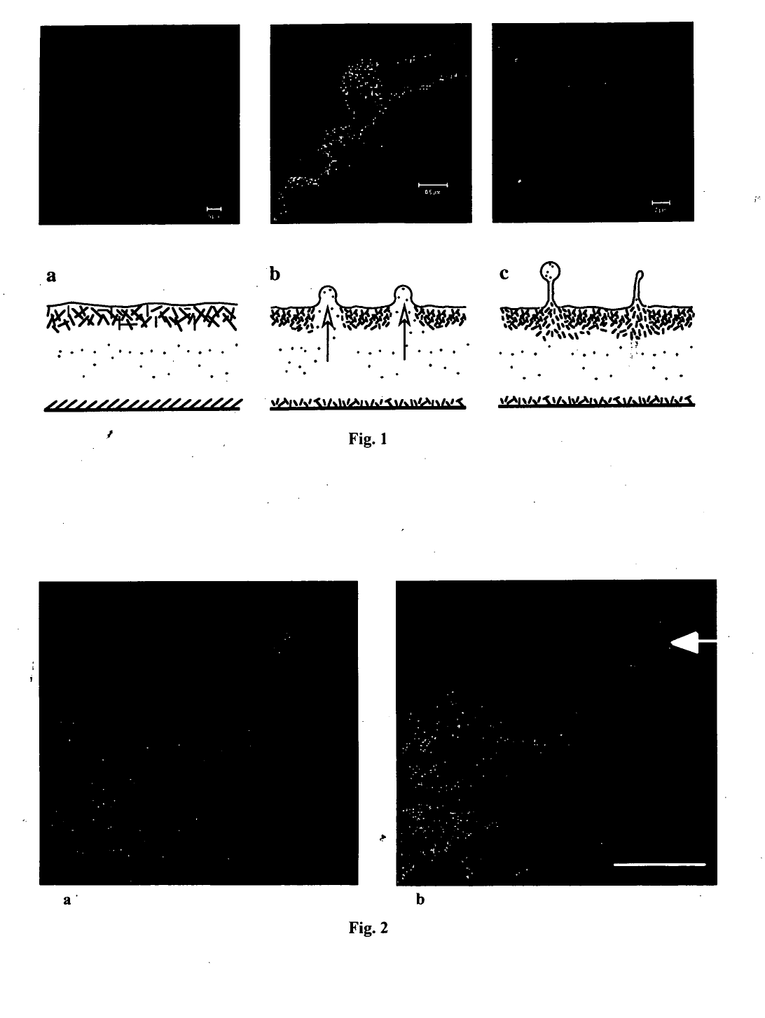

FIG. 2: Confocal image (excitation: 488 nm; emission: 510 nm) illustrating the incorporation of parts of the endoplasmic reticulum into “native” vesicles ((a) image at the fluorescence wavelength; (b) image at the fluorescence wavelength superimposed on transmission image). The endoplasmic reticulum was visualized by the expression of recombinant EGFP (Enhanced GFP; Clontech, Palo Alto, Calif., USA) in HEK293 cells.

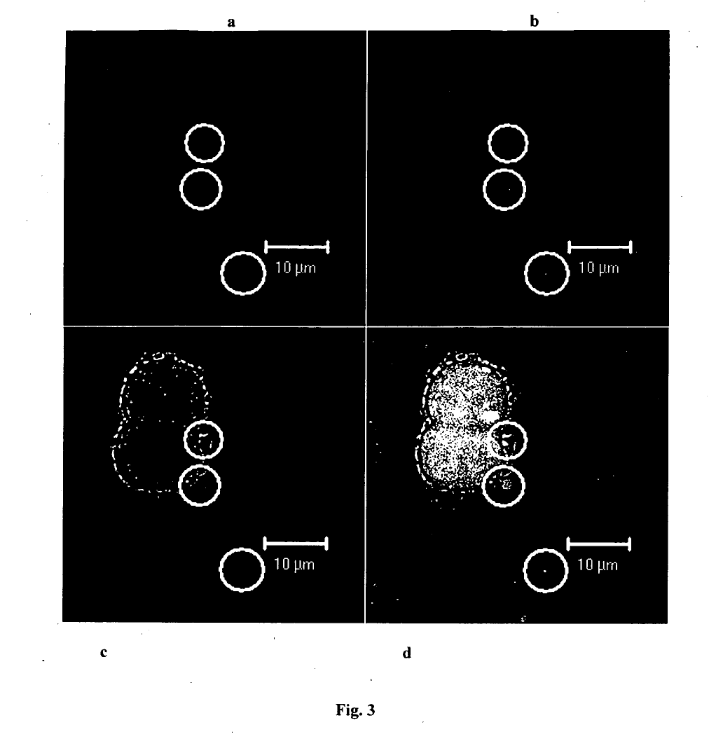

The EGFP here was furnished with a signal sequence which guides the fluorescent protein during its expression into the lumen of the endoplasmic reticulum (ER), thus “staining” the ER. The arrow marks a vesicle that was pinched off from a living HEK293 cell after the addition of cytochalasin B [10 μg/ml]. The bar bottom right corresponds to a length of 5 μm.

FIG. 3: Confocal image of a dividing HEK cell treated for vesicle formation, which expresses heterologously GFP-labeled G_{α} protein. The cell had been treated beforehand with the calcium indicator Fura Red. Shown here in clockwise order, starting top left, are: (a) Fura Red signal, (b) GFP-G_{α} signal, (c) transmission image, (d) superimposition of images (a) and (c). The Fura Red signal (image (a)) was recorded using a high-pass filter (cut-off filter with transmission for λ>650 nm). Image (b) shows the fluorescence emission of the GFP between 500 and 530 nm. The transmission image corresponds to a NORMARSKI image. For all the images shown, fluorescence excitation took place at 488 nm.—“Native” vesicles, budding vesicles and those which have already pinched off are shown with circles. Note that the vesicles are carrying both the recombinant product (Ga protein) and the calcium indicator.

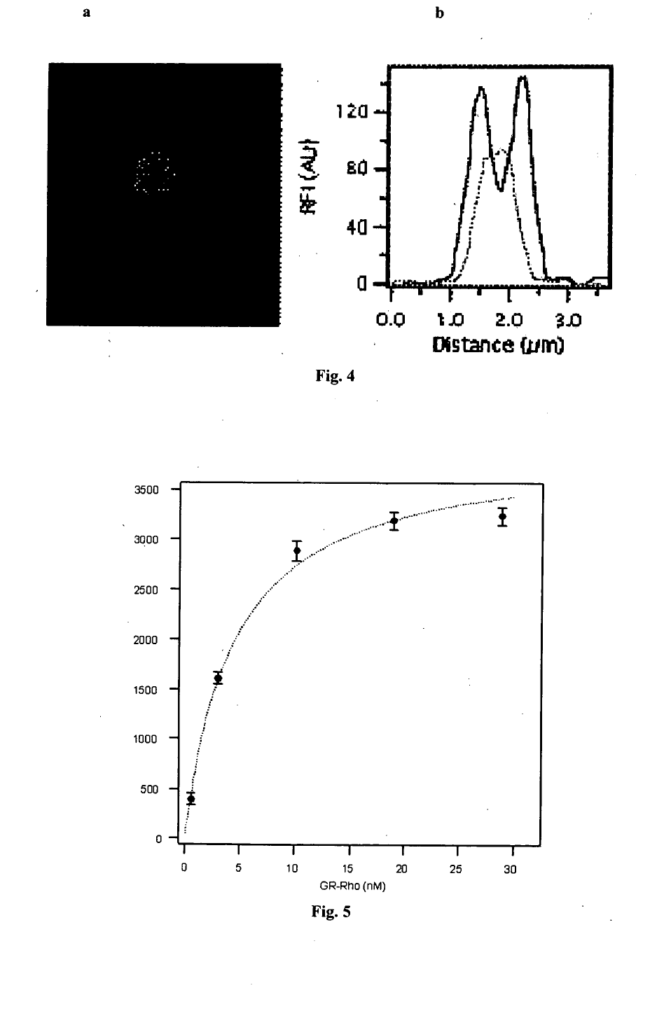

FIG. 4: (a) Fluorescence of a vesicle produced from a living HEK293 cell with GFP-labeled cytosolic cell contents carried over from the cell of origin (green fluorescence in vesicle interior) and 5HT3 receptor incorporated in the vesicle membrane, labeled with GR-Cy5 (red fluorescence from the outer region of the vesicle). (b) Line profiles of green and red fluorescence.

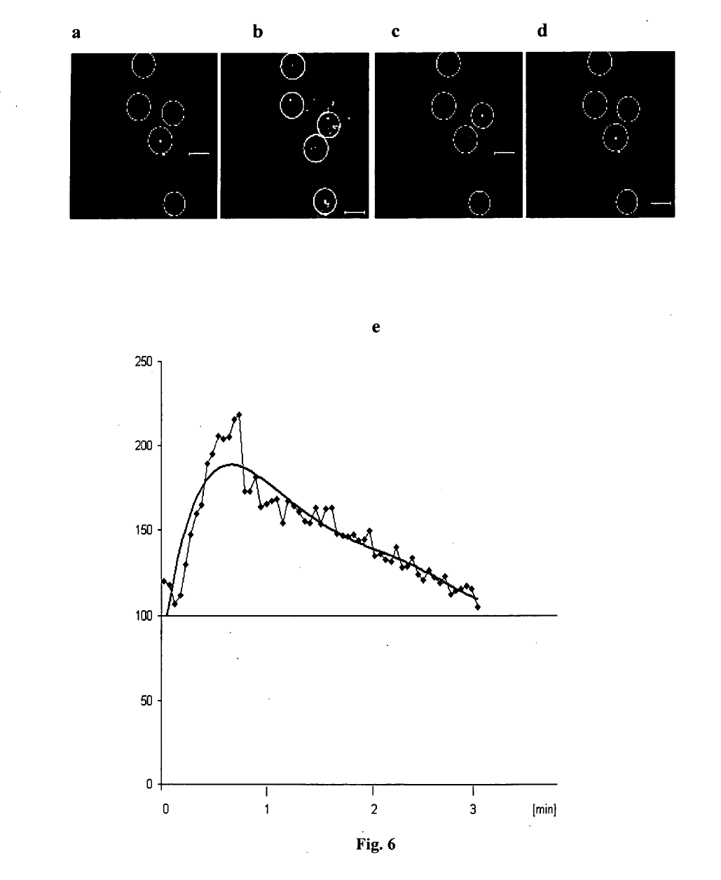

FIG. 5: Binding curve of a fluorescent ligand bound to individual discrete vesicles (n=3) was determined by sequential addition of ascending concentrations of the fluorescent ligand to one and the same cover glass with vesicles immobilized thereon by overnight incubation.

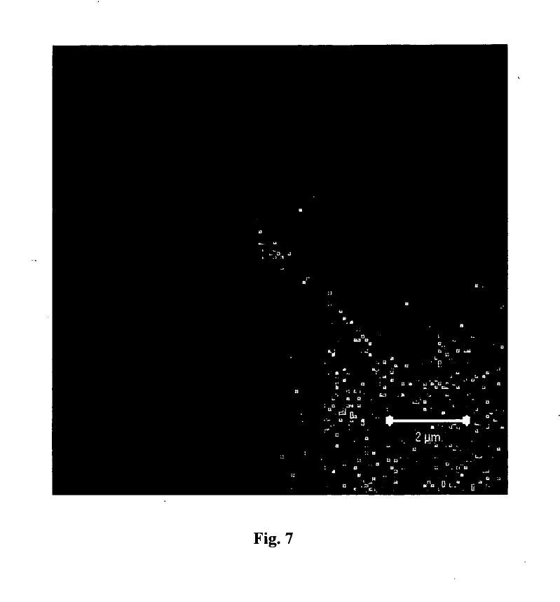

Fluorescence intensities F as a function of ascending concentrations of GR-Rho, presented as fluorescent ligand of the 5HT₃ receptor incorporated in “native” vesicles from HEK293 cells (after subtraction of the fluorescence intensity F₀ in the absence of GR-Rho). The fit of measurement data with the Langmuir binding isotherm equation gives a value of K_{d}=(4.2+/−1.7) nM for the dissociation constant of the system comprising the HT₃ receptor and the GR-Rho ligand.

FIG. 6: Demonstration of the ability of the serotonin 5-HT₃ receptor incorporated in vesicles to regulate the concentration of intravesicular calcium ions, visualized on the basis of the fluorescence of the calcium indicator Fluo-3 (excitation: 488 nm; emission: 500-530 nm). Stimulation of vesicles loaded with Fluo-3 by addition of 350 μM m-chlorophenylbiguanide hydrochloride (mCPG), as a receptor-specific agonist.

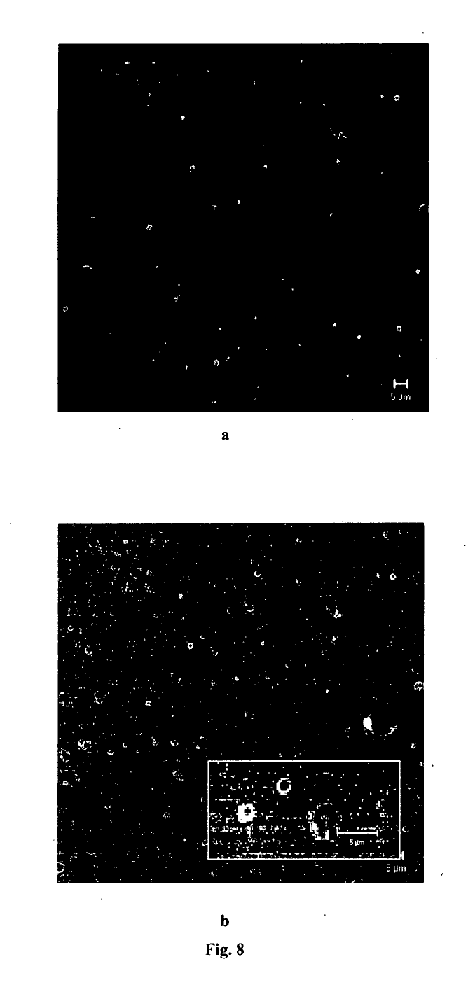

Confocal false-color images before and t=0 (a, b), 45 sec (c) and 3 min (d) after addition of the serotonin agonist. Shot (b) presents the analyzed image sections (circles) against the background of the transmission image, superimposed with the fluorescence of the calcium indicator not coded with false colors. Note that the fluorescence signals encompass a larger area than the vesicles in view of the high degree of enhancement.

(e) Time curve of the fluorescence signal of a vesicle which showed the strongest Ca response. The stimulation was performed by addition of the agonist (mCPG) to the vesicle medium (t=0 sec). The maximum fluorescence signal that occurred through binding of the calcium to the calcium indicator Fluo-3 was already measured after less than one minute.

FIG. 7: A “native” vesicle becoming detached from an HEK293 cell, which expresses the GFP-labeled NK1 receptor. The NK1 receptor protein here was fused with the Aequorea victoria GFP at the level of the coding DNA.

FIG. 8: Confocal images illustrating the quality of “native” vesicles which have been stored for 30 days at −80° C. and then thawed out. “Native” vesicles were produced by cytochalasin B treatment of HEK293 cells which express the transiently transfected, recombinant EGFP (Clontech; Palo Alto, Calif., USA) in the cytosol. (a) Fluorescence image (excitation 488 nm; emission 510 nm); (b) combination of transmission image and fluorescence image. The section at bottom right shows a greater magnification of intact “native” vesicles.

1-112. (canceled).

113. A bioanalytical reagent with at least one vesicle, generated from a living cell, comprising at least one receptor, characterized in that a mechanism of signal transduction triggered by said receptor in the cell used for vesicle generation is preserved in said vesicle as a component of the bioanalytical reagent.

114. A bioanalytical reagent with at least one vesicle, generated from a living cell, according to claim 113, wherein the vesicle as a component of said reagent comprises further cell products and/or cell proteins, besides said one or more receptors, which are involved in said mechanism of signal transduction, besides said one or more receptors.

115. A bioanalytical reagent with at least one vesicle, generated from a living cell, according to claim 113, wherein said one or more vesicles are generated from a eukaryotic cell.

116. A bioanalytical reagent with at least one vesicle, generated from a living cell, according to claim 113, wherein said one or more vesicles are generated from a cell of a native tissue.

117. A bioanalytical reagent with at least one vesicle, generated from a living cell, according to claim 113, wherein the interior of said one or more vesicles is free from cell nucleus material, so that replication processes do not occur within said one or more vesicles.

118. A bioanalytical reagent with at least one vesicle, generated from a living cell, according to claim 113, wherein said one or more vesicles have a diameter of 50 nm-5000 nm, preferably of 100 nm-2000 nm.

119. A bioanalytical reagent with at least one vesicle, generated from a living cell, according to claim 113, wherein the one or more receptors are present in natural form in said one or more vesicles as a component of the bioanalytical reagent.

120. A bioanalytical reagent with at least one vesicle, generated from a living cell, according to claim 113, wherein the one or more receptors are present in a modified form in said one or more vesicles as a component of the bioanalytical reagent.

121. A bioanalytical reagent with at least one vesicle, generated from a living cell, according to claim 113, wherein the one or more receptors are present in a form resulting from a recombinant fabrication process in said one or more vesicles as a component of the bioanalytical reagent.

122. A bioanalytical reagent with at least one vesicle, generated from a living cell, according to claim 113, comprising the preservation of a binding capability of said one or more receptors to a specific ligand, this binding capacity being present in said vesicle-generating cell and the receptor being associated with the vesicle as a component of the bioanalytical reagent.

123. A bioanalytical reagent with at least one vesicle, generated from a living cell, according to claim 113, wherein the one or more receptors are selected from the group of signal-transducing receptors that is formed by plasma membrane receptors, such as ion channel receptors, G protein-coupled receptors (GPCR), orphan receptors, enzyme-coupled receptors, such as receptors with an intrinsic tyrosine kinase activity, receptors with an intrinsic serine/threonine kinase activity, furtheron by receptors for growth factors (peptide hormone receptors), receptors for chemotactic substances, such as the class of chemokine receptors, and by intracellular hormone receptors, such as steroid hormone receptors.

124. A bioanalytical reagent with at least one vesicle, generated from a living cell, according to claim 113, wherein said one or more vesicles comprise, besides said one or more receptors, further biological compounds (components) from the group that is formed, e.g., by G-proteins and G-protein regulators (e.g. rasGAP), enzymes, such as adenylate cyclases, phospholipases forming intracellular secondary messenger compounds (e.g. cAMP (cyclic adenosine monophosphate), cGMP (cyclic guanosine monophosphate), diacyl glycerol (DAG) or inositol triphosphate (IP3)), enzymes, such as serine, threonine and tyrosine kinases, and tyrosine phosphatases that activate or inhibit proteins by phosphorylation or de-phosphorylation.

125. A bioanalytical reagent with at least one vesicle, generated from a living cell, according to claim 113, wherein biological, biochemical or synthetic compounds like cell surface proteins or cell surface sugars are associated with the outer membrane of the one or more vesicles, which associated compounds are used for the transport of said vesicle to pre-determined destinations, such as cells and/or organs and/or pre-determined tissue in a living organism, and/or for the binding to a biological or biochemical or synthetic recognition element, which specifically recognizes and binds said biological or biochemical or synthetic recognition element.

126. A bioanalytical reagent with at least one vesicle, generated from a living cell, according to claim 113, wherein the mechanism of signal transduction comprises a mechanism from among the group of mechanisms that is formed e.g. from ion conducting, G-protein coupling, activation or inhibition of intra-vesicular ion channels, intra-vesicular release of calcium, protein activation or inhibition by enzymatic phosphorylation or de-phosphorylation (kinase cascades; phosphatases), and release or enzymatic formation of secondary messenger compounds, such as cAMP, cGMP or diacyl glycerol (DAG), inositol triphosphate (IP3).

127. A bioanalytical reagent with at least one vesicle, generated from a living cell, according to claim 113, wherein said mechanism of signal transduction comprises a (secondary) functional response of the one or more vesicle-associated receptors after a primary specific interaction of said one or more receptors with one or more natural and/or synthetic ligands contained in a sample that is brought into contact with said vesicle.

128. A bioanalytical reagent with at least one vesicle, generated from a living cell, according to claim 113, wherein said mechanism of signal transduction comprises the activation of an ion channel of a receptor associated with a vesicle, as a component of said bioanalytical reagent.

129. A bioanalytical reagent with at least one vesicle, generated from a living cell, according to claim 113, wherein said mechanism of signal transduction comprises the binding of a G-protein to a receptor associated with a vesicle, as a component of said bioanalytical reagent.

130. A bioanalytical reagent with at least one vesicle, generated from a living cell, according to claim 113, wherein said mechanism of signal transduction comprises the internal release of ions, such as Ca²⁺, or of other messenger compounds, such as cAMP or cGMP.

131. A bioanalytical reagent with at least one vesicle, generated from a living cell, according to claim 113, wherein said mechanism of signal transduction comprises the enzymatic decomposition of a substrate to a product by a vesicle-associated enzyme.

132. A bioanalytical reagent with at least one vesicle, generated from a living cell, according to claim 127, wherein a (secondary) functional response as part of said mechanism of signal transduction occurs after interaction between one or more natural and/or synthetic ligands or co-factors contained in a sample brought into contact with said vesicle on the one hand and naturally or recombinantly generated proteins associated with said vesicle on the other.

133. A bioanalytical reagent with at least one vesicle, generated from a living cell, according to claim 113, wherein said one or more vesicles additionally comprise components for generation of an experimentally detectable signal.

134. A bioanalytical reagent with at least one vesicle, generated from a living cell, according to claim 133, wherein said additional components for generation of an experimentally detectable signal are associated with the further biological compounds (components) according to claim 128, these further biological compounds (components) being associated with the one or more vesicles as a component of said bioanalytical reagent.

135. A bioanalytical reagent with at least one vesicle, generated from a living cell, according to claim 133, wherein said additional components for generation of an experimentally detectable signal are associated with a receptor.

136. A bioanalytical reagent with at least one vesicle, generated from a living cell, according to claim 133, wherein said additional components for generation of an experimentally detectable signal are parts of fusion proteins that are associated with said one or more vesicles.

137. A bioanalytical reagent with at least one vesicle, generated from a living cell, according to claim 133, wherein said additional components for generation of an experimentally detectable signal are selected from the group of components formed by absorptive indicators and luminescent indicators, luminescence labels, luminescent nanoparticles, absorptive indicator proteins and luminescent indicator proteins, such as BFP (“blue fluorescent protein”), GFP (“green fluorescent protein”) or RFP (“red fluorescent protein”), artificial luminescent amino acids, radioactive labels, spin labels, such as NMR labels or ESR labels, ion indicators, especially pH and calcium indicators, or potential-dependent indicators, such as potential-dependent luminescence labels, or redox complexes.

138. A bioanalytical reagent with at least one vesicle, generated from a living cell, according to claim 133, wherein said additional components for generation of an experimentally detectable signal are generated from the same cell from which the vesicle was generated.

139. A bioanalytical reagent with at least one vesicle, generated from a living cell, according to claim 133, wherein said additional components for generation of an experimentally detectable signal are inserted into the vesicle after its formation.

140. A bioanalytical reagent with at least one vesicle, generated from a living cell, according to claim 113, wherein the functionality of a receptor associated with a vesicle being a component of said bioanalytical reagent is preserved upon storage under deep-frozen conditions for a period of at least one week, preferably of at least one month, especially preferred for at least one year.

141. A bioanalytical reagent with at least one vesicle, generated from a living cell, according to claim 113, wherein it is characterized by a shelf life of at least one week under sterile conditions in cooled buffer solution, i.e. at a temperature below ambient temperature, e.g. at 4° C.

142. A method for production of a bioanalytical reagent with a vesicle generated from a living cell, according to claim 113, wherein said vesicle was produced from a living cell comprising at least one receptor, and wherein a mechanism of signal transduction triggered by said receptor in said living cell is preserved in said vesicle as a component of the bioanalytical reagent.

143. A method for production of a bioanalytical reagent with a vesicle generated from a living cell, according to claim 142, wherein the constriction of said vesicle from said living cell is effected after application of cytochalasin B and/or cytochalasin D.

144. A method according to claim 142, wherein said method comprises the application of shear forces and/or of centrifugation steps, for example upon exposure to a gradient of sucrose, and/or the application of chromatographic steps, for example by separation into fractions of different size distributions, and/or the application of filtration steps and/or the application of electrophoretic methods.

145. A method according to claim 142, wherein the interior of a vesicle produced by said method is free from cell nucleus material, so that replicative processes do not occur.

146. A method according to claim 142, comprising the preservation of a binding capability of said one or more receptors to a specific ligand, this binding capability being present in said vesicle-generating cell and the receptor being associated with the vesicle as a component of the bioanalytical reagent.

147. A method according to claim 142, wherein the one or more receptors are selected from the group of signal-transducing receptors that is formed by plasma membrane receptors, such as ion channel receptors, G protein-coupled receptors (GPCR), orphan receptors, enzyme-coupled receptors, such as receptors with an intrinsic tyrosine kinase activity, receptors with an intrinsic serine/threonine kinase activity, furtheron by receptors for growth factors (peptide hormone receptors), receptors for chemotactic substances, such as the class of chemokine receptors, and by intracellular hormone receptors, such as steroid hormone receptors.

148. A method according to claim 142, wherein said one or more vesicles produced by this method comprise, besides said one or more receptors, further biological compounds (components) from the group that is formed e.g. by G-proteins and G-protein regulators (e.g. rasGAP), enzymes, such as adenylate cyclases, phospholipases forming intracellular secondary messenger compounds (e.g. cAMP (cyclic adenosine monophosphate), cGMP (cyclic guanosine monophosphate), diacyl glycerol (DAG) or inositol triphosphate (IP3)), enzymes, such as serine, threonine, and tyrosine kinases, and tyrosine phosphatases that activate or inhibit proteins by phosphorylation or de-phosphorylation.

149. A method according to claim 142, wherein biological, biochemical or synthetic compounds, such as cell surface proteins or cell surface sugars, are associated with the outer membrane of the one or more vesicles produced by this method, these compounds being used for the transport of said vesicle to pre-determined destinations, such as cells and/or organs and/or pre-determined tissue in a living organism, and/or for the binding to a biological or biochemical or synthetic recognition element, which specifically recognizes and binds said biological or biochemical or synthetic recognition element.

150. A method according to claim 142, wherein said one or more vesicles produced by this method additionally comprise components for generation of an experimentally detectable signal.

151. A method for production of a bioanalytical reagent with a vesicle generated from a living cell, according to claim 142, wherein said vesicle is merged with an artificial lipid vesicle to form a mixed vesicle.

152. A method according to claim 151, wherein said mixed vesicle is substantially enlarged in comparison to the vesicle generated from a living cell, for example to a diameter of 5 μm-50 μm.

153. A method according to claim 151, wherein said mixed vesicle comprises additional natural and/or artificial lipids and/or also additional proteins with additional functionalities, in comparison to the vesicle generated from a living cell.

154. A bioanalytical detection method with a bioanalytical reagent according to claim 113, wherein said detection method is selected from the group that is formed, for example, by optical detection methods, such as refractrometric methods, surface plasmon resonance, optical absorption measurements (e.g. internal reflection methods using a highly refractive material, in combination with infrared spectroscopic measurements) or luminescence detection (e.g. fluorescence correlation spectroscopy), detection of energy or charge transfer, mass spectroscopy, electrical or electrochemical detection methods, such as electrophysiology, patch clamp techniques, impedance measurements, electronic resonance measurements, such as electron spin resonance or nuclear spin resonance, gravimetric methods (e.g. electrical crystal balance measurements), radioactive methods, or by electrophoretic measurements.

155. A bioanalytical detection method according to claim 154, wherein said method is performed in homogeneous solution.

156. A bioanalytical detection method according to claim 154, wherein said method is performed using a measurement arrangement with at least 2 electrodes and separate compartments adequate for receiving liquids, wherein a solid carrier (preferably as an electrically isolating separation wall), comprising at least one aperture and separating at least 2 compartments, is located between two electrodes facing each other, the electrodes being of any geometrical form and each extending into at least one compartment or being in contact with at least one compartment.

157. A bioanalytical detection method according to claim 156, wherein said measurement arrangement is provided with means on one side or on both sides of the carrier which enable a supply of liquid and/or a storage of liquid and/or an exchange of liquid and/or the addition of vesicles generated from a living cell, from a bioanalytical reagent according to the invention, between carrier and electrode.

158. A bioanalytical detection method according to claim 156, wherein the one or more apertures of said measurement arrangement have such a diameter that, in the presence of a potential difference over the measurement arrangement and mediated by the two or more electrodes, an inhomogeneous electrical field is generated around the aperture, said field having an increasing value with decreasing distance from the aperture and said field being capable of moving vesicles electrophoretically towards the aperture, said vesicles being located close to said aperture and generated from a living cell, from a bioanalytical reagent according to the invention.

159. A bioanalytical detection method according to claim 156, wherein the one or more apertures of said measurement arrangement have such a diameter that vesicles generated from a living cell, from a bioanalytical reagent according to the invention, can be positioned over or within the aperture by means of a hydrodynamic or electrokinetic flow or by other mechanical manipulation (e.g. by means of optical tweezers, force microscope or by a micro manipulator).

160. A bioanalytical detection method according to claim 156, wherein the carrier of said measurement arrangement is provided with an electrically charged surface which exerts attractive force on vesicles generated from a living cell, from a bioanalytical reagent according to the invention, or is provided with an adhesion promoting layer for binding said vesicles on its surface.

161. A bioanalytical detection method according to claim 156, wherein vesicles generated from a living cell, from a bioanalytical reagent according to the invention, are inserted between separation wall or carrier and electrode into a compartment filled or not filled with buffer beforehand, and wherein said vesicles are moved towards the aperture by means of an electrical potential difference applied to the electrodes, or are positioned on the aperture by hydrodynamic or electrokinetic flow and/or are positioned on the aperture mechanically (e.g. by means of optical tweezers, force microscope or by a micro manipulator).

162. A bioanalytical detection method according to claim 156, wherein vesicles generated from a living cell, from a bioanalytical reagent according to the invention, are positioned on said aperture, the vesicle membranes form an electrically close contact with the carrier over the aperture, and a measurement of the (electrical) membrane resistance is enabled.

163. A bioanalytical detection method according to claim 156, wherein artificial lipid vesicles with a diameter larger than the diameter of said aperture are added to at least one compartment, in order to generate a planar lipid bilayer on the surface of the carrier and extending over the aperture, and wherein then vesicles generated from a living cell, from a bioanalytical reagent according to the invention are added to said compartment, in order to merge said vesicles with the generated lipid membrane and to make receptors that are associated with said vesicles generated from living cells accessible for electrical or optical measurements.

164. A bioanalytical detection method according to claim 156, wherein membrane proteins are inserted into a vesicle generated from a living cell, after positioning said vesicle on an aperture.

165. A bioanalytical detection method according to claim 156, wherein a vesicle generated from a living cell located over an aperture or a planar membrane generated from said vesicle and spanning an aperture is accessible for optical measurements, especially for fluorescence measurements, or for simultaneous optical and electrical measurements, to which it is subjected.

166. A bioanalytical detection method according to claim 156, wherein a measurement arrangement or a measurement system with several apertures on one carrier is used, and wherein measurements on at least two apertures are performed sequentially and/or in parallel.

167. A bioanalytical detection method according to claim 156, wherein a multitude of vesicles generated from living cells, from a bioanalytical reagent according to the invention, is arranged in an array on a solid, electrically isolating carrier, wherein said array of vesicles is brought into electrically isolating contact with an array of patch-clamp pipets in a geometrical arrangement similar to that of the vesicle array, in order to enable a simultaneous performance of electrical measurements independently of each other or simultaneous electrical and optical measurements on a multitude of individual vesicles.

168. A bioanalytical detection method according to claim 154, wherein the at least one vesicle generated from a living cell, comprising at least one receptor, from a bioanalytical reagent according to the invention, is immobilized on the surface of a solid support.

169. A bioanalytical detection method according to claim 168, wherein a mechanism of a signal transduction triggered by said receptor in said living cell is retained in a vesicle generated from the cell after immobilization of the vesicle.

170. A bioanalytical detection method with at least one vesicle immobilized on the surface of a solid support, the vesicle being generated from a living cell, from a bioanalytical reagent according to claim 113, comprising at least one receptor, wherein a mechanism of a signal transduction triggered by said receptor in said living cell is preserved in a vesicle generated from the cell after immobilization of the vesicle.

171. A bioanalytical detection method according to claim 170, wherein vesicles, each comprising at least one receptor, are immobilized in discrete measurement areas (d) with one or more vesicles each on the surface of said solid support.

172. A bioanalytical detection method according to claim 170, wherein vesicles with at least two different kinds of receptor are immobilized in a multitude of measurement areas (d), wherein preferably within an individual measurement area always vesicles with a uniform kind of receptor are immobilized.

173. A bioanalytical detection method according to claim 168, wherein the immobilization of the one or more vesicles generated from a living cell, on the surface of said solid support, is performed upon covalent binding or upon physical adsorption (electrostatic or van-der-Waals interaction or hydrophilic or hydrophobic interaction or a combination of these interactions).

174. A bioanalytical detection method according to claim 167, wherein an adhesion-promoting layer is deposited between the surface of said solid support and the one or more vesicles immobilized thereon.

175. A bioanalytical detection method according to claim 174, wherein the adhesion-promoting layer comprises a chemical compound of the group of silanes, epoxides, functionalized, charged or polar polymers and “self-organized functionalized mono or multiple layers”.

176. A bioanalytical detection method according to claim 174, wherein the adhesion-promoting layer comprises a monomolecular layer of mainly one kind of protein, such as serum albumins or streptavidin, or of modified proteins, such as biotinylated serum albumin.

177. A bioanalytical detection method according to claim 174, wherein the adhesion-promoting layer comprises self-organized alkane-terminated monolayers of mainly one kind of chemical or biochemical molecules.

178. A bioanalytical detection method according to claim 174, comprising association with the adhesion-promoting layer of biological or biochemical or synthetic recognition elements which recognize and bind a vesicle generated from a living cell with surface-associated biological or biochemical or synthetic components for specific recognition and binding from the bioanalytical reagent, wherein biological, biochemical or synthetic compounds like cell surface proteins or cell surface sugars are associated with the outer membrane of the one or more vesicles, which associated compounds are used for the transport of said vesicle to pre-determined destinations, such as cells and/or organs and/or pre-determined tissue in a living organism, and/or for the binding to a biological or biochemical or synthetic recognition element, which specifically recognizes and binds said biological or biochemical or synthetic recognition element.

179. A bioanalytical detection method according to claim 154, wherein at least one ligand for a receptor, which is bound to a vesicle generated from a living cell, from a bioanalytical reagent according to the invention, is immobilized, optionally by means of a spacer molecule, on the surface of the solid support.

180. A bioanalytical detection method according to claim 179, wherein at least two different ligands for receptors, which are bound to a vesicle generated from a living cell, from a bioanalytical reagent according to the invention, are immobilized in a multitude of measurement areas (d), wherein preferably a uniform kind of ligand is immobilized within an individual measurement area.

181. A bioanalytical detection method according to claim 179, wherein said ligands are immobilized on the surface of the solid support upon covalent binding or upon physical adsorption (e.g. electrostatic or van-der-Waals interaction or hydrophilic or hydrophobic interaction or a combination of these interactions).

182. A bioanalytical detection method according to claim 179, wherein an adhesion-promoting layer is applied between the surface of the solid support and said ligands immobilized thereon.

183. A bioanalytical detection method according to claim 171, wherein regions between the laterally separated measurement areas, with vesicles, generated from living cells (from a bioanalytical reagent according to the invention) immobilized in these measurement areas, or with ligands for receptors that are bound to vesicles generated from living cells (from a bioanalytical reagent according to the invention), and/or regions within these measurement areas, between the compounds immobilized therein, are “passivated” in order to minimize non-specific binding of analytes or of their detection reagents, i.e., that compounds which are “chemically neutral” towards the analyte are deposited between the laterally separated measurement areas (d) and/or within these measurement areas (d) between said immobilized compounds, the “chemically neutral” compounds preferably being composed of the groups that are formed by albumins, casein, detergents, such as Tween 20, detergent/lipid mixtures (of synthetic and/or natural lipids), synthetic and natural lipids or also hydrophilic polymers, such as polyethylene glycols or dextrans.

184. A bioanalytical detection method according to claim 156, wherein the material of the solid support (carrier) with immobilized vesicles, generated from living cells (from a bioanalytical reagent according to the invention), or with immobilized ligands for receptors that are bound to vesicles generated from living cells (from a bioanalytical reagent according to the invention), comprises a material of the group which is formed e.g. by moldable, sprayable or millable plastics, carbon compounds, metals, such as gold, silver, copper, metal oxides or silicates, such as glass, quartz or ceramics, or silicon or germanium or ZnSe or a mixture of these materials.

185. A bioanalytical detection method according to claim 156, wherein the surface of said solid support (carrier) is essentially planar.

186. A bioanalytical detection method according to claim 156, wherein said solid support (carrier) is an optical or electronic sensor platform.

187. A bioanalytical detection method according to claim 156, wherein said solid support (carrier) is transparent at least in a region of wavelengths in the ultraviolet to infrared spectrum and comprises preferably a material from the group that is formed e.g. by moldable, sprayable or millable plastics, carbon compounds, metals, metal oxides or silicates, such as glass, quartz or ceramics, or silicon or germanium or ZnSe or a mixture of these materials.

188. A bioanalytical detection method according to claim 186, wherein said solid support is an optical waveguide used as a sensor platform.

189. A bioanalytical detection method according to claim 186, wherein said solid support is an optical thin-film waveguide used as a sensor platform, with an initial optically transparent layer (a) with refractive index n1 on a second optically transparent layer (b) with refractive index n₂, wherein n₂, wherein n, >n₂.

190. A bioanalytical detection method according to claim 188, wherein the sensor platform as a solid support is divided into two or more discrete waveguiding regions.

191. A bioanalytical detection method according to claim 189, wherein the material of the second optically transparent layer (b) of the sensor platform as a solid support is selected from the group that is formed by silicates, such as glass or quartz, or transparent moldable, sprayable or millable, especially thermoplastic plastics, such as polycarbonates, polyimides, polymethyl methacrylates, or polystyrenes.

192. A bioanalytical detection method according to claim 189, wherein the refractive index of the first optically transparent layer (a) of the sensor platform as a solid support is greater than 1.8.

193. A bioanalytical detection method according to claim 189, wherein the first optically transparent layer (a) comprises a material of the group of TiO₂, ZnO, Nb₂O₅, Ta₂O₅, HfO₂, or ZrO₂, preferably of TiO₂ or Ta₂O₅ or Nb₂O₅.

194. A bioanalytical detection method according to claim 189, wherein an additional optically transparent layer (b′) with lower refractive index than layer (a) and with a thickness of 5 nm-10 000 nm, preferably of 10 nm-1000 nm, is located between the optically transparent layers (a) and (b) and in contact with layer (a).

195. A bioanalytical detection method according to claim 189, wherein the in-coupling of excitation light into the optically transparent layer (a) to the measurement areas (d) on the sensor platform as a solid support is performed using one or more optical in-coupling elements from the group formed by prism couplers, evanescent couplers comprising joined optical waveguides with overlapping evanescent fields, butt-couplers with focusing lenses, preferably cylindrical lenses, arranged in front of a front face (distal end) of the waveguiding layer, and grating couplers.

196. A bioanalytical detection method according to claim 189, wherein the in-coupling of excitation light into the optically transparent layer (a) to the measurement areas (d) is performed using one or more grating structures (c) that are formed in the optically transparent layer (a).

197. A bioanalytical detection method according to claim 156, wherein one or more liquid samples, comprising vesicles generated from living cells (from a bioanalytical reagent according the invention), with associated receptors, are brought into contact with the ligands for these receptors, immobilized in one or more measurement areas, and wherein a signal change caused by a binding of the receptors associated with said vesicles to their immobilized ligands is measured.

198. A bioanalytical detection method according to claim 197, wherein the signal transduction of receptors associated with vesicles generated from living cells (from a bioanalytical reagent according to the invention) after binding of these receptors to their immobilized ligands, is measured, wherein this signal transduction can be triggered, for example, by binding of further ligands to the receptors associated with said vesicles, or by other inducing influences.

199. A bioanalytical detection method according to claim 197, wherein the binding of receptors that are associated with vesicles generated from living cells (from a bioanalytical reagent according to the invention) to said immobilized ligands occurs in competition with the binding of these receptors associated with said vesicles to ligands in free solution.

200. A bioanalytical detection method according to claim 156, wherein one or more liquid samples are brought into contact with the vesicles, which are generated from living cells (from a bioanalytical reagent according to the invention) and immobilized in one or more measurement areas, along with their associated receptors, and wherein a signal change resulting from the binding of ligands to said receptors or from other inducing influences on said receptors is measured.

201. A bioanalytical detection method according to claim 156, wherein one or more liquid samples are brought into contact with the vesicles, which are generated from living cells (from a bioanalytical reagent according to the invention) and immobilized in one or more measurement areas, along with their associated receptors, and wherein the signal transduction of those receptors resulting from the binding of ligands to said receptors or from other inducing influences on said receptors is measured.

202. A bioanalytical detection method according to claim 200, wherein the binding of ligands from a supplied sample to receptors that are associated with the immobilized vesicles generated from living cells (from a bioanalytical reagent according to the invention) occurs in competition with the binding of these ligands to receptors in free solution which are optionally associated with vesicles.

203. A bioanalytical detection method according to claim 197, wherein one or more liquid samples, comprising vesicles generated from living cells (from a bioanalytical reagent according to the invention) with associated receptors, are brought into contact with the ligands for these receptors, the ligands being immobilized in one or more measurement areas, excitation light from one or more light sources of similar or different wavelengths is in-coupled to the measurement areas (d) by one or more grating structures (c), and the change of optical signals emanating from one or more measurement areas (d), caused by a binding of the receptors associated with said vesicles to their immobilized ligands, is measured.

204. A bioanalytical detection method according to claim 200, wherein one or more liquid samples are brought into contact with the vesicles immobilized in one or more measurement areas (d), along with their associated receptors, excitation light from one or more light sources of similar or different wavelengths is in-coupled to the measurement areas (d) by one or more grating structures (c), and the change of optical signals emanating from one or more measurement areas (d), caused by the binding of the ligands to said receptors or by other inducing influences on said receptors, is measured.

205. A bioanalytical detection method according to claim 204, wherein said changes of optical signals from the measurement areas (d) are caused by changes of the effective refractive index in the near-field of the optically transparent layer (a) in these measurement areas and are measured at the actual excitation wavelength.

206. A bioanalytical detection method according to claim 204, wherein said changes of optical signals from the measurement areas (d) are changes of one or more luminescences of similar or different wavelength, which have been excited in said measurement areas in the near-field of the optically transparent layer (a), and which are measured each at a wavelength different from the corresponding excitation wavelength.

207. A bioanalytical detection method according to claim 204, wherein the one or more luminescences and/or measurements of light signals at the excitation wavelength are determined polarization-selectively, wherein preferably the one or more luminescences are measured at a polarization that is different from the polarization of the excitation light.

208. A bioanalytical detection method according to claim 154, for the simultaneous or sequential, quantitative and/or qualitative determination of one or more analytes from the group of receptors or ligands, chelators or “histidine tag components”, enzymes, enzyme co-factors or inhibitors.

209. A bioanalytical detection method according to claim 154, wherein the samples to be examined are, for example, aqueous solutions or surface water or soil or plant extracts or bio- or process broths, or are taken from biological tissue fractions or from food, or odorous or flavoring substances or cosmetic compounds.

210. The use of a vesicle as a component of a bioanalytical reagent according to claim 113 for the enrichment of membrane receptors or for the enrichment of proteins (such as antigens) triggering an immunological response in a two- or three-dimensional phase, which can then e.g. be administered to living organisms (e.g. to stimulate immune defense processes).

211. The use of a vesicle, as a component of a bioanalytical reagent according to claim 113 as a compartment for therapeutic, diagnostic, photosensitive or other biologically active compounds for administration to a living organism.

212. The use of a bioanalytical reagent according to claim 113 for investigating receptor-ligand interactions, especially for determining the binding strength and kinetic parameters of these interactions between a receptor and its ligand, or for determining the channel activity of an ion channel receptor after ligand binding or other inducing influences on said receptor, or for determining the enzymatic activity of enzymes associated with a vesicle, as a component of a bioanalytical reagent according to the invention, or for determining secondary messenger compounds after ligand binding to a receptor resulting in a signal transduction, or for determining protein-protein interactions, or for determining protein kinases.

213. The use of a bioanalytical reagent according to claim 113 for quantitative and/or qualitative analyses for determining chemical, biochemical or biological analytes in screening methods in pharmaceutical research, combinatorial chemistry, clinical and pre-clinical development, for real-time binding studies and for determining kinetic parameters in affinity screening and in research, for qualitative and quantitative analyte determinations, especially for DNA- and RNA analytics, for generation of toxicity studies and for the determination of expression profiles, and for determining antibodies, antigens, pathogens or bacteria in pharmaceutical product development and research, human and veterinary diagnostics, agrochemical product development and research, for symptomatic and pre-symptomatic plant diagnostics, for patient stratification in pharmaceutical product development and for therapeutic drug selection, for determining pathogens, nocuous agents and germs, especially of salmonella, prions and bacteria, in food and environmental analytics, and for analysis and quality control of odorous and flavoring substances.

214. The use of a bioanalytical detection method according to claim 154 for investigating receptor-ligand interactions, especially for determining the binding strength and kinetic parameters of these interactions between a receptor and its ligand, or for determining the channel activity of an ion channel receptor after ligand binding or other inducing influences on said receptor, or for determining the enzymatic activity of enzymes associated with a vesicle, as a component of a bioanalytical reagent according to the invention, or for determining secondary messenger compounds after ligand binding to a receptor resulting in a signal transduction, or for determining protein-protein interactions, or for determining protein kinases.

215. The use of a bioanalytical detection method according to claim 154 for quantitative and/or qualitative analyses for determining chemical, biochemical or biological analytes in screening methods in pharmaceutical research, combinatorial chemistry, clinical and pre-clinical development, for real-time binding studies and for determining kinetic parameters in affinity screening and in research, for qualitative and quantitative analyte determinations, especially for DNA- and RNA analytics, for generation of toxicity studies and for the determination of expression profiles, and for determining antibodies, antigens, pathogens or bacteria in pharmaceutical product development and research, human and veterinary diagnostics, agrochemical product development and research, for symptomatic and pre-symptomatic plant diagnostics, for patient stratification in pharmaceutical product development and for therapeutic drug selection, for determining pathogens, nocuous agents and germs, especially of salmonella, prions and bacteria, in food and environmental analytics, and for analysis and quality control of odorous and flavoring substances.
